SEQUENCE LISTING (28 sequences)

SEQ ID NO: 1 - DNA, 27 bp, Mus musculus
agcccatgtttctgcatggatttccca

SEQ ID NO: 2 - DNA, 31 bp, Artificial sequence
  synthetic sequence
gtttttgggtacaaagacgtacctaaagagc

SEQ ID NO: 3 - DNA, 27 bp, Homo sapiens
atcccacgtttttgcatgaatttatag

SEQ ID NO: 4 - DNA, 21 bp, Artificial sequence
  synthetic sequence
cgtaccccgattcaggtgatg

SEQ ID NO: 5 - DNA, 20 bp, Artificial sequence
  synthetic sequence
gccggatttagctctgctct

SEQ ID NO: 6 - DNA, 20 bp, Artificial sequence
  synthetic sequence
cgccaccagttcgccatgga

SEQ ID NO: 7 - DNA, 20 bp, Artificial sequence
  synthetic sequence
tacagcccggggagcatcgt

SEQ ID NO: 8 - DNA, 18 bp, Artificial sequence
  synthetic sequence
gatgtcgctcgaccgctg

SEQ ID NO: 9 - DNA, 20 bp, Artificial sequence
  synthetic sequence
cggtacaatgtagacggcga

SEQ ID NO: 10 - DNA, 58 bp, Artificial sequence
  synthetic sequence
ccggcccatgtttctgcatggatttctcgagaaatccatgcagaaacatgggtttttg

SEQ ID NO: 11 - DNA, 58 bp, Artificial sequence
  synthetic sequence
ccggccttaccactatcagagagtactcgagtactctctgatagtggtaaggtttttg

SEQ ID NO: 12 - DNA, 58 bp, Artificial sequence
  synthetic sequence
ccggctgtccaatgtcaaccggaatctcgagattccggttgacattggacagtttttg

SEQ ID NO: 13 - DNA, 19 bp, Artificial sequence
  synthetic sequence
gggaagcccatcaccatct

SEQ ID NO: 14 - DNA, 20 bp, Artificial sequence
  synthetic sequence
gcctcaccccatttgatgtt

SEQ ID NO: 15 - DNA, 25 bp, Artificial sequence
  synthetic sequence
tgaggccggtgctgagtatgtcgtg

SEQ ID NO: 16 - DNA, 23 bp, Artificial sequence
  synthetic sequence
tccttggaggccatgtaggccat

SEQ ID NO: 17 - DNA, 25 bp, Artificial sequence
  synthetic sequence
ctggctacctgagtgaagatggaga

SEQ ID NO: 18 - DNA, 25 bp, Artificial sequence
  synthetic sequence
aaagacctcccctccgatgtagtag

SEQ ID NO: 19 - DNA, 20 bp, Artificial sequence
  synthetic sequence
acacattgggagaggtgtgc

SEQ ID NO: 20 - DNA, 20 bp, Artificial sequence
  synthetic sequence
gcaagggtccattcaggtgt

SEQ ID NO: 21 - DNA, 20 bp, Artificial sequence
  synthetic sequence
gccttaccactatcagagag

SEQ ID NO: 22 - DNA, 20 bp, Artificial sequence
  synthetic sequence
aaccttacctcatcagagag

SEQ ID NO: 23 - DNA, 19 bp, Artificial sequence
  synthetic sequence
aactctccaatgtcaaccg

SEQ ID NO: 24 - DNA, 18 bp, Artificial sequence
  synthetic sequence
gctgtccaatgtcaaccg

SEQ ID NO: 25 - DNA, 20 bp, Artificial sequence
  synthetic sequence
tcatttcagagggcaccacc

SEQ ID NO: 26 - DNA, 18 bp, Artificial sequence
  synthetic sequence
caacttcttctccccgcc

SEQ ID NO: 27 - DNA, 20 bp, Artificial sequence
  synthetic sequence
tgtatcttgccgttcccacc

SEQ ID NO: 28 - DNA, 20 bp, Artificial sequence
  synthetic sequence
ctccacagtgaccacactcc

(19) United States
(12) Patent Application Publication
(10) Pub. No.: US 2020/0155638 A1
(43) Pub. Date: May 21, 2020

(54) Enhancing Oxytocin Receptor Expression Using an Oxytocin Receptor Agonist and an Alk5 Antagonist

(71) Applicant: The Regents of the University of California, Oakland, CA (US)

(72) Inventors: Irina M. Conboy, El Sobrante, CA (US); Michael J. Conboy, El Sobrante, CA (US)

(21) Appl. No.: 16/615,353

(22) PCT Filed: May 29, 2018
(86) PCT No.: PCT/US2018/034907

§ 371 Date: Nov. 20, 2019

Related U.S. Application Data
(60) Provisional application No. 62/512,566, filed on May 30, 2017.

(51) Int. Cl. A61K 38/095; A61K 38/17

(52) U.S. Cl. CPC A61K 38/095; A61K 38/179

(57) ABSTRACT

Methods are provided for treating a subject with an effective amount of an oxytocin receptor (OXTR) agonist and an effective amount of an ALK5 antagonist (ALK5i) to enhance OXTR expression in the subject. Methods are also provided for treating a subject with an effective amount of an OXTR agonist and an effective amount of an ALK5i to enhance hippocampal neurogenesis, enhance functional learning and reduce senescence in a tissue of the subject. In certain aspects, the amounts of the OXTR agonist and ALK5i may be sufficient to protect tissue maintenance and repair during acute and chronic viral infections. In certain aspects, the amount of the OXTR agonist and ALK5 antagonist may be sufficient to provide the subject with a sense of psychological well-being.

### CROSS-REFERENCE TO RELATED APPLICATIONS

This application claims the benefit of U.S. Provisional Patent Application No. 62/512,566 filed May 30, 2017, which application is incorporated herein by reference in its entirety.

### GOVERNMENT RIGHTS

This invention was made with government support under contract AG048316 awarded by the National Institute of Health/National Institute on Aging. The Government has certain rights in the invention.

### INTRODUCTION

As the average world population is aging rapidly, enhancing the elderly quality of life is of major importance for both their well-being and for regulating the associated socioeconomic costs. With aging, the capacity of tissues to regenerate declines and eventually fails, leading to degenerative disorders and eventual organ failure. As people age they may also become more susceptible to viral infections.

The inventors have found that unrelated viral pathologies have a common consequence, in that levels of oxytocin receptors are diminished. Viral infections in general may also play a role in decreasing muscle health and regeneration, a decline in metabolic health, and a lower sense of wellbeing, as these rely on effective OXTR signaling.

While several treatments for the health and maintenance of old tissues have been tested, most of them fail in elderly populations and the risk to benefit ratio is so high that exercise is still the primary treatment for the age-specific muscle wasting.

### SUMMARY

Methods are provided for treating a subject with an effective amount of an oxytocin receptor (OXTR) agonist and an effective amount of an ALK5 antagonist (ALK5i) to enhance OXTR expression in the subject. Methods are also provided for treating a subject with an effective amount of an OXTR agonist and an effective amount of an ALK5i to enhance hippocampal neurogenesis, enhance functional learning and reduce senescence in a tissue of the subject. In certain aspects, the OXTR agonist may be oxytocin, an oxytocin analog (e.g., carbetocin, demoxytocin, TC OT 39, WAY 267464, or a small molecule). The ALK5 antagonist may be a small molecule, such as A 83-01, D 4476, GW 788388, LY 364947, RepSox, SB 431542, SB 505124, SB 525334, or SD 208. In certain aspects, the amounts of the OXTR agonist and ALK5i may be sufficient to protect tissue maintenance and repair during acute and chronic viral infections. In certain aspects, the amount of the OXTR agonist and ALK5 antagonist may be sufficient to provide the subject with a sense of psychological well-being.

Aspects of the methods disclosed here include treating a subject with a viral infection. The method may include treating the subject having a viral infection with an oxytocin receptor (OXTR) agonist and ALK5 antagonist, wherein the amount of the OXTR agonist and ALK5 antagonist is effective to enhance OXTR expression in the subject.

The subject may be a human. Alternatively, the subject may be an animal for example, a rodent or a primate. The viral infection may be an acute viral infection. Alternatively, the viral infection may be a chronic viral infection. In certain aspects the effective amount of an OXTR agonist and ALK5 antagonist are administered sim ultaneously.

In addition to possessing a viral infection, the subject may also be old or suffering from reduction in muscle mass or neurons, or reduction in other tissues, such as a reduction caused due to natural aging process, injury, extended inactivity, disease, and the like. The subject may be diagnosed as having or susceptible to developing a neurodegenerative disease, such as, Alzheimer's disease, Parkinson's disease, Huntington's disease, or dementia, or CNS inflammation. The subject may be diagnosed as having or susceptible to developing a muscular degeneration. The subject may be suffering from muscular dystrophy due to disease or muscular atrophy due to inactivity associated with an injury or disease. The subject may be diagnosed as having or susceptible liver fibrosis or adiposity or reduced liver regeneration.

The amount of the OXTR agonist for the administration step, e.g., for administering to a subject may be in the range of 7.5 nM-30 nM and the amount of the ALK5 antagonist may be in the range of 0.05 μM-3 μM. The ratio of OXTR agonist to the ALK5 antagonist used for the administration step, e.g., for administering to a subject as disclosed herein may be 1:50, 50:1, 1:40, 40:1, 1:30, 30:1, 1:25, 25:1, 1:10, 10:1, 1:5, 5:1, or 1:1.

In certain aspects, the OXTR agonist may be oxytocin. In certain aspects, the ALK5 antagonist may be 2-(3-(6-Methylpyridin-2-yl)-1H-pyrazol-4-yl)-1,5-naphthyridine.

In certain aspects, the OXTR expression in the subject may be as compared to the OXTR expression from a healthy adult subject.

In certain aspects, the method may further comprising assessing the OXTR expression in the subject following the administration and adjusting the amount of the OXTR agonist and/or the ALK5 antagonist for the next administration step.

Administering the OXTR agonist and the ALK5 antagonist to a subject may be systemic or local and may be continuous or on an administration schedule, such as, bi-daily, daily, bi-weekly, weekly, bi-monthly, or monthly.

In certain aspects, the method may further include assessing the OXTR expression in the subject following the administration and increasing or decreasing the amount of the OXTR agonist.

In certain aspects, the method may further include assessing the OXTR expression in the subject following the administration and increasing or decreasing the amount of the ALK5 antagonist.

In certain aspects, the method may further include assessing the OXTR expression in the subject following the repeated administration and adjusting the administration schedule.

In another aspect the methods disclosed here include enhancing hippocampal neurogenesis in a subject. The method may include treating the subject with an oxytocin receptor (OXTR) agonist and ALK5 antagonist, wherein the amount of the OXTR agonist and ALK5 antagonist is effective to reduce CD45+ cells in the brain of the subject. The hippocampal neurogenesis of the subject may exhibit a two-fold increase within 1 week. In certain aspects, the CD45+ cells are in the brain of an aged subject and are comparable to the CD45+ cells in the brain of a younger subject.

In yet another aspect the methods disclosed here include enhancing functional learning in a subject. The method may include treating the subject with an oxytocin receptor (OXTR) agonist and ALK5 antagonist, wherein the amount of the OXTR agonist and ALK5 antagonist is effective to improve memory and cognition in the subject.

In still another aspect the methods disclosed here include reducing senescence (p16) in at least one tissue of a subject. The method may include treating the subject with an oxytocin receptor (OXTR) agonist and ALK5 antagonist, wherein the amount of the OXTR agonist and ALK5 antagonist is effective to reduce p16 in an at least one old muscle, the liver or the brain of the subject. In certain aspects the p16 is reduced in at least one tissue of the subject as compared to the p16 in the same type of tissue from a younger subject.

In still another aspect the methods disclosed here include reducing liver adiposity and fibrosis in a subject. The method may include treating the subject with an oxytocin receptor (OXTR) agonist and ALK5 antagonist, wherein the amount of the OXTR agonist and ALK5 antagonist is effective to reduce liver adiposity and/or lifer fibrosis and enhance liver regeneration.

### BRIEF DESCRIPTION OF THE DRAWINGS

FIG. 1, panels A and B, shows that ALK5i and an OXTR agonist enhances myogenesis and reduces fibrosis in old mice in vivo.

FIG. 2 shows that ALK5i and an OXTR agonist avoids Alk5i-promoted down regulation of OXTR.

FIG. 3, panels A-D, shows that liver regeneration and health improve in old mice administered with ALK5i and an OXTR agonist.

FIG. 4, panels A-F, shows that ALK5i and an OXTR agonist, but not each molecule alone quickly enhances hippocampal neurogenesis in old mice.

FIG. 5 shows that learning is improved in old mice treated with ALK5i and an OXTR agonist.

FIG. 6, panels A-E, shows that ALK5i and an OXTR agonist attenuate p16 in muscle, liver and brain.

FIG. 7 shows the in vivo immunofluorescence of p16 and eMyHC in young injured/regenerating muscle.

FIG. 8, panels A and B, shows that OXTR expression is down regulated upon lentiviral transduction.

FIG. 9 shows complete shRNA 1, 2, 3 and mix data for in vitro mice.

FIG. 10 shows myoblasts. vs. satellite cells, vs. control shRNA satellite cells in vitro.

FIG. 11, panel A shows that OXTR downregulation depends on dosage of viral transduction for no-target shRNA and empty vector particles.

FIG. 11, panel B shows time point data for downregulated OXTR expression for non-target shRNA and empty vector particles.

FIG. 12 shows that OXTR downregulation upon lentiviral transduction is evolutionarily conserved between mice and human.

FIG. 13 shows that high sequence homology is observed between human and mouse Smad3 loci in the area targeted by shRNA.

FIG. 14, panels A and B, shows that human database reveals down regulation of OXTR expression upon difference viral transfections.

FIG. 15, panels A-E, shows that viral transductions or infections do not universally down-regulate cell surface receptors.

FIG. 16 shows that lentiviral transfection down regulates OXTR as determined by the immunofluorescence.

FIG. 17, panels A and B, shows that lentiviral transduction decreases myogenic activity in primary mouse myoblasts.

### DETAILED DESCRIPTION

Methods are provided for treating a subject with an effective amount of an oxytocin receptor (OXTR) agonist and an effective amount of an ALK5 antagonist. In certain aspects, the OXTR agonist may be oxytocin, an oxytocin analog (e.g., carbetocin, demoxytocin, TC OT 39, WAY 267464), or another small molecule. The ALK5 antagonist may be a small molecule, such as 2-(3-(6-Methylpyridin-2-yl)-1H-pyrazol-4-yl)-1,5-naphthyridine, A 83-01, D 4476, GW 788388, LY 364947, RepSox, SB 431542, SB 505124, SB 525334, or SD 208. In certain aspects, the amounts of the OXTR agonist and ALK5 antagonist may be sufficient to enhance OXTR expression in the subject. In certain aspects, the amounts of the OXTR agonist and ALK5 antagonist may be sufficient to enhance hippocampal neurogenesis in the subject. In certain aspects the amounts of the OXTR agonist and ALK5 antagonist may be sufficient to enhance functional learning in the subject. In certain aspects the amounts of the OXTR agonist and ALK5 antagonist may be sufficient to and reduce senescence in a tissue of the subject.

Before the present methods are further described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The terms “treatment”, “treating” and the like as used herein refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease.

An “inhibitor” or “antagonist” as used herein refers to any agent (e.g., small molecule, macromolecule, peptide, etc.) that reduces the activity of an enzyme or receptor. A “Competitive inhibitor” as used herein refers to an inhibitor that reduces binding of a substrate to an enzyme or receptor, such as the binding of a ligand to a sell surface receptor. The competitive inhibitor may specifically bind to the active site of the enzyme or an allosteric site of the enzyme, or may specifically bind the substrate itself. “Non-competitive inhibitor” as used herein refers to an inhibitor that reduces activity of an enzyme regardless of the presence of the substrate. A non-competitive inhibitor may bind to an active site of the enzyme or to an allosteric site of the enzyme.

As used herein, an “oxytocin analog” refers to a peptide having a similar amino acid sequence to oxytocin, with one or more amino acid substitutions, unnatural amino acids, side chain modifications, or any other suitable modification.

The terms “individual,” “subject,” “host,” and “patient,” are used interchangeably herein and refer to any mammalian subject for whom diagnosis, treatment, or therapy is desired, particularly humans.

The terms “specific binding,” “specifically binds,” and the like, refer to the preferential binding of a binding element (e.g., one binding pair member to the other binding pair member of the same binding pair) relative to other molecules or moieties in a solution or reaction mixture. The binding element may specifically bind (e.g., covalently or non-covalently) to a particular epitope or narrow range of epitopes within the cell. In certain aspects, the binding element non-covalently binds to a target.

The term “effective amount” as used herein refer to the amount of an agent (e.g., dosage, concentration in plasma, etc.) that elicits a desired biological effect, such as enhancing or suppressing the signaling of a cell surface receptor (e.g., OXTR or ALK5i) or inducing tissue regeneration (e.g., muscle regeneration). Effective amounts may readily be determined empirically from assays, from safety and escalation and dose range trials, individual clinician-patient relationships, as well as in vitro and in vivo assays such as those described in the art.

By “IC50” is intended the concentration of an antagonist required to achieve 50% inhibition of a specific biological or biochemical function, such as ALK5 signaling. By “EC50” is intended the plasma concentration required for obtaining 50% of a maximum biological effect.

The term “sarcopenia” as used herein refers to the degenerative loss of skeletal muscle mass and/or strength, and is associated with aging. In contrast, “muscle regeneration” as used herein refers to the increase in muscle (e.g., skeletal muscle) mass or strength upon treatment.

The term “neurogenesis” as used herein refers to the generation of new neurons in adult mammalian brain (primarily, but not exclusively, in hippocampus, region of brain responsible for learning and memory).

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### OXTR Agonists

The oxytocin receptor (OXTR) is a G-protein coupled receptor for the peptide oxytocin, which acts a hormone and neurotransmitter. Oxytocin is FDA approved and sold under the name of Pitocin and Syntocinon. Aspects of the invention include an OXTR agonist (such as oxytocin) or the use thereof.

An OXTR agonist is any agent that specifically enhances OXTR expression, OXTR signaling, or signaling downstream of the OXTR. In certain aspects, the OXTR agonist may include oxytocin (e.g., Pitocin, Syntocinon or generic oxytocin) or an oxytocin mimetic, i.e., a peptide having a similar amino acid sequence to oxytocin, one or more amino acid substitutions, unnatural amino acids, or any other suitable modification. An oxytocin analog of the subject invention may be 8 or 9 amino acids in length. An oxytocin analog may have one or more, two or more, three or more, or four or more chemical modifications as compared to oxytocin.

Examples of oxytocin analogs that act as OXTR agonists include Demoxytocin, Carbetocin, TC OT 39 and WAY-267464 (e.g., WAY- 267464 dihydrochloride), or a derivative thereof. Demoxytocin (also known as Sandopart or deaminooxytocin) is an analogue of oxytocin, and an OXTR agonist. Demoxytocin has an IUPAC name of 2-[(1-{[13-(butan-2-yl)-10-(2-carbamoylethyl)-7-(carbamoylmethyl)-16-[(4- hydroxyphenyl)methyl]-6,9,12,15,18-pentaoxo-1,2-dithia-5,8,11,14,17-pentaazacycloicosan-4-yl]carbonyl}pyrrolin-2-yl)formamido]-N-(carbamoylmethyl)-4-methylpentanamide and a Chemical Abstracts Service registry number (CAS number) of 113-78-0. Carbetocin (also known as Duratocin, Pabal, Lonactene) is an eight amino acid long oxytocin analogue that primarily agonizes peripherally expressed oxytocin receptors. Carbetocin has an IUPAC name of (2S)-1-[(3S,6S,9S,12S,15S)-12-[(2S)-butan-2-yl]-9-(2-carbamoylethyl)-6-carbamoylmethyl)- 15-[(4-hydroxyphenyl)methyl]-16-methyl-5,8,11,14,17-pentaoxo-1-thia-4,7,10,13,16-pentazacycloicosane-3-carbonyl]-N-[(1S)-1-(carbamoylmethylcarbamoyl)- 3-methyl-butyl]pyrrolidine-2-carboxamide and a CAS number of 37025-55-1. TC OT 39 is a non-peptide oxytocin analog and partial agonist of OXTR and vasopressin V2 receptors. TC OT 39 has an IUPAC name of (2S)-N-[[4-[(4,10-Dihydro-1-methylpyrazolo[3,4-b][1,5]benzod iazepin-5(1H)-yl)carbonyl]-2-methylphenyl]methyl]-2-[(hexahydro-4- methyl-1H-1,4-diazepin-1-yl)thioxomethyl]-1-pyrrolidinecarboxamide and a CAS number of 479232-57-0. WAY-267464 is a non-peptide oxytocin analogue and OXTR agonist with minimal affinity for vasopressin receptors. WAY-267464 has an IUPAC name of 4-(3,5-dihydroxybenzyl)-N-(2-methyl-4-[(1-methyl-4,10-dihydropyrazolo[3, 4-b][1,5]benzodiazepin-5(1H)-yl)carbonyl]benzyl)piperazine-1-carboxamide and a CAS number of 847375-16-0.

In certain aspects, the OXTR agonist may be a small molecule. For example, the OXTR agonist may be 1 kDa or less, 900 Da or less, 800 Da or less, 700 Da or less, 600 Da or less, 500 Da or less, 400 Da or less, 300 Da or less, 200 Da or less, or 100 Da or less. Small molecule compounds may be dissolved in water or alcohols or solvents such as DMSO or DMF, and diluted into water or an appropriate buffer prior to being provided to cells. The OXTR agonist may optionally include a moiety preventing transport across the blood brain barrier (BBB).

OXTR agonists are well known in the art, as evidenced by U.S. Pat. No. 8,748,564 and US Publication Nos. US20070117794 and US20130085106, the disclosures of which are incorporated herein by reference.

In certain aspects, the OXTR agonists may include an OXTR specific binding member. The terms “specific binding,” “specifically binds,” and the like, refer to the preferential binding of a domain (e.g., one binding pair member to the other binding pair member of the same binding pair) relative to other molecules or moieties in a solution or reaction mixture. The binding domain may specifically bind (e.g., covalently or non-covalently) to a particular epitope or narrow range of epitopes within the cell. In such instances, the OXTR specific binding member association with OXTR may be characterized by a KD (dissociation constant) of 10⁻⁵ M or less, 10⁻⁶ M or less, such as 10⁻⁷ M or less, including 10⁻⁶ M or less, e.g., 10⁻⁶ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less.

A variety of different types of specific binding members may be employed. Binding members of interest include, but are not limited to, antibodies, proteins, peptides, haptens, nucleic acids, aptamers, etc. In certain aspects, the OXTR specific binding member may be an antibody or a fragment thereof. The term “antibody” as used herein includes polyclonal or monoclonal antibodies or fragments thereof that are sufficient to bind to an analyte of interest. The fragments can be, for example, monomeric Fab fragments, monomeric Fab′ fragments, or dimeric F(ab)′2 fragments. Also within the scope of the term “antibody” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

In certain embodiments, the OXTR agonist may be an agent that modulates, e.g., enhances, expression of functional OXTR. OXTR expression may be enhanced using any convenient means, including use of an agent that enhances OXTR expression, such as, but not limited to vectors (e.g., plasmids, retroviruses, etc.) encoding functional OXTR under an inducible promoter, tissue specific promoter, or may be constitutively expressed.

### ALK5 Antagonists

Activin A receptor type II-like kinase (ALK5), also known as transforming growth factor beta receptor I (TGF-β receptor), is a serine/threonine kinase receptor expressed in a variety of tissues. Alk5 inhibitors are undergoing several clinical trials for treating cancers and attenuating metastasis. Certain aspects of the invention include an ALK5 antagonist (i.e., an ALK5 inhibitor, ALK5i) or the use thereof.

An ALK5 antagonist is any agent that specifically reduces OXTR expression, ALK5 signaling, or signaling downstream of the OXTR. Examples of ALK5 agonists include competitive inhibitors and non-competitive inhibitors. An ALK5 agonist may bind TGF-β or the TGF-β receptor.

In certain aspects, the ALK5 antagonist may be a small molecule. For example, the ALK5 antagonist may be 1 kDa or less, 900 Da or less, 800 Da or less, 700 Da or less, 600 Da or less, 500 Da or less, 400 Da or less, 300 Da or less, 200 Da or less, or 100 Da or less. Small molecule compounds may be dissolved in water or alcohols or solvents such as DMSO or DMF, and diluted into water or an appropriate buffer prior to being provided to cells. The small molecule may be a competitive inhibitor of ALK5-TGF-β binding or a non-competitive inhibitor of ALK5 activity.

Examples of ALK5 antagonists include 2-(3-(6-Methylpyridin-2-yl)-1H-pyrazol-4-yl)-1,5-naphthyridine, galunisertib (LY2157299 monohydrate), A 83-01, D 4476, GW 788388, LY 364947, RepSox, SB 431542, SB 505124, SB 525334, and SD 208, or a derivative thereof. A 83-01 is a selective inhibitor of ALK4, ALK5 and ALK7. A 83-01 has an IUPAC name of 3-(6-Methyl-2-pyridinyl)-N-phenyl-4-(4-quinolinyl)-1H-pyrazole-1-carbothioamide and a Chemical Abstracts Service registry number (CAS number) of 909910-43-6. D 4476 is an inhibitor of ALK5 and CK1. D 4476 has an IUPAC name of 4-[4-(2,3-Dihydro-1,4-benzodioxin-6-yl)-5-(2-pyridinyl)-1H-imidazol-2-yl]benzamide and a CAS number of 301836-43-1. GW 788388 is a selective inhibitor of ALK5. GW 788388 has an IUPAC name of 4-[4-[3-(2-Pyridinyl)-1 H-pyrazol-4-yl]-2-pyridinyl]-N-(tetrahydro-2H-pyran-4-yl)-benzamide and a CAS number of 452342-67-5. LY 364947 (also known as HTS 466284) is a selective inhibitor of ALK5. LY 364947 has an IUPAC name of 4-[3-(2-Pyridinyl)-1H-pyrazol-4-yl]-quinoline and a CAS number of 396129-53-6. RepSox (also known as E-616452 and SJN 2511) is a selective inhibitor of ALK5. RepSox has an IUPAC name of 2-(3-(6-Methylpyridine-2-yl)-1H-pyrazol-4-yl)-1,5-naphthyridine and a CAS number of 446859-33-2. SB 431542 is a selective inhibitor of ALK4, ALK5 and ALK7. SB 431542 has an IUPAC name of 4-[4-(1,3-benzodioxol-5-yl)-5-(2-pyridinyl)-1H-imidazol-2-yl]benzamide and a CAS number of 301836-41-9. SB 505124 is a selective inhibitor of ALK4, ALK5 and ALK7. SB 505124 has an IUPAC name of 2-[4-(1,3-Benzodioxol-5-yl)-2-(1,1-dimethylethyl)-1H-imidazol-5-yl]-6-methyl-pyridine and a CAS number of 694433-59-5. SB 525334 is a selective inhibitor of ALK5. SB 525334 has an IUPAC name of 6-[2-(1,1-Dimethylethyl)-5-(6-methyl-2-pyridinyl)-1H-imidazol-4-yl]quinoxaline and a CAS number of 356559-20-1. SD 208 is an ATP-competitive ALK5 inhibitor. SD 208 has an IUPAC name of 2-(5-Chloro-2-fluorophenyl)-4-[(4-pyridyl)amino]pteridine and a CAS number of 627536-09-8.

ALK5 antagonists are well known in the art, as evidenced by US Publication Nos. US20060194845, US20050245520, and US20040266842, the disclosures of which are incorporated herein by reference.

In certain aspects, the ALK5 antagonists may include an ALK5 specific binding member. The terms “specific binding,” “specifically binds,” and the like, refer to the preferential binding of a domain (e.g., one binding pair member to the other binding pair member of the same binding pair) relative to other molecules or moieties in a solution or reaction mixture. The binding domain may specifically bind (e.g., covalently or non-covalently) to a particular epitope or narrow range of epitopes within the cell. In such instances, the ALK5 specific binding member association with ALK5 may be characterized by a KD (dissociation constant) of 10⁻⁵ M or less, 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less.

A variety of different types of specific binding members may be employed. Binding members of interest include, but are not limited to, antibodies, proteins, peptides, haptens, nucleic acids, aptamers, etc. In certain aspects, the ALK5 specific binding member may be an antibody or a fragment thereof. The term “antibody” as used herein includes polyclonal or monoclonal antibodies or fragments thereof that are sufficient to bind to an analyte of interest. The fragments can be, for example, monomeric Fab fragments, monomeric Fab′ fragments, or dimeric F(ab)′2 fragments. Also within the scope of the term “antibody” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

In certain embodiments, the ALK5 antagonist may be an agent that modulates, e.g., inhibits, expression of functional ALK5. Inhibition of ALK5 expression may be accomplished using any convenient means, including use of an agent that inhibits ALK5 expression, such as, but not limited to: antisense agents, RNAi agents, agents that interfere with transcription factor binding to a promoter sequence of the ALK5 gene, or inactivation of the ALK5 gene, e.g., through recombinant techniques, etc.

For example, antisense molecules can be used to down-regulate expression of ALK5 in the cell. The anti-sense reagent may be antisense oligodeoxynucleotides (ODN), such as synthetic ODN having chemical modifications from native nucleic acids, nucleic acid constructs that express such anti-sense molecules as RNA, and so forth. The antisense sequence may be complementary to the mRNA of the targeted protein (i.e., ALK5). Antisense molecules inhibit gene expression through various mechanisms, e.g., by reducing the amount of mRNA available for translation, through activation of RNAse H, or steric hindrance. One or a combination of antisense molecules may be administered, where a combination may include multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule may be a synthetic oligonucleotide. Antisense oligonucleotides may be at least 7 nucleotides in length, at least 10 nucleotides in length, at least 15 nucleotides in length, at least 20 nucleotides in length, 500 or fewer nucleotides in length, 100 or fewer nucleotides in length, 50 or fewer nucleotides in length, 25 or fewer nucleotides in length, between 7 and 50 nucleotides in length, between 10 and 25 nucleotides in length, and so forth, where the length is governed by efficiency of inhibition, specificity, including absence of cross-reactivity, and the like.

A specific region or regions of the endogenous sense strand mRNA sequence may be chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Oligonucleotides may be chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

In addition, the transcription level of an ALK5 can be regulated by gene silencing using RNAi agents, e.g., double-strand RNA (Sharp (1999) Genes and Development 13: 139-141). RNAi, such as double-stranded RNA interference (dsRNAi) or small interfering RNA (siRNA), has been extensively documented in the nematode C. elegans (Fire, A., et al, Nature, 391, 806-811, 1998) and routinely used to “knock down” genes in various systems. RNAi agents may be dsRNA or a transcriptional template of the interfering ribonucleic acid which can be used to produce dsRNA in a cell. In these embodiments, the transcriptional template may be a DNA that encodes the interfering ribonucleic acid. Methods and procedures associated with RNAi are also described in WO 031010180 and WO 01/68836, all of which are incorporated herein by reference. dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety). A number of options can be utilized to deliver the dsRNA into a cell or population of cells such as in a cell culture, tissue, organ or embryo. For instance, RNA can be directly introduced intracellularly. Various physical methods are generally utilized in such instances, such as administration by microinjection (see, e.g., Zernicka-Goetz, et al. (1997) Development 124:1133-1137; and Wianny, et al. (1998) Chromosoma 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

### Methods of Treatment

Aspects of the invention are directed to a method of treating a subject. In certain aspects the subject has a viral infection. The method may include administering an oxytocin receptor (OXTR) agonist and an ALK5 antagonist to the subject. As described above, the OXTR agonist may be any agent that specifically enhances OXTR expression, OXTR signaling, or signaling downstream of the OXTR and the ALK5 antagonist may be any agent that specifically reduces ALK5 expression or ALK5 signaling. In certain aspects the OXTR agonist may be oxytocin. In certain aspects the ALK5 antagonist may be galunisertib (LY2157299 monohydrate). The amount of the OXTR agonist and ALK5 antagonist administered in the subject methods may be effective to achieve any of a number of desired outcomes, as discussed below.

A combination of oxytocin (or another OXTR agonist) with Alk5 inhibitor is expected to potentiate the positive effects on the OXTR receptor, hippocampal neurogenesis, functional learning and senescence in a tissue.

The amount of the OXTR agonist and ALK5 antagonist may therefore be effective to enhance OXTR expression, enhance muscle repair, enhance cognition, reduce memory loss, enhance liver regeneration, brain-neurogenesis, reduce neuro-inflammation, restore productive hematopoiesis and/or broadly improve tissue maintenance and repair as well as a sense of psychological well-being in an elderly subject with a viral infection. Unlike many other substances that are postulated to enhance tissue regeneration, oxytocin is not associated with cancers, e.g. is not oncogenic and Alk5 inhibitors are in fact anti-oncogenic and are in clinical trials for suppressing metastasis (Inman, G. J. (2011). Switching TGFbeta from a tumor suppressor to a tumor promoter. Current opinion in genetics & development 21, 93-99.). Thus, a combination of oxytocin (or another OXTR agonist) with Alk5 inhibitor will be used to enhance the OXTR expression and therefore improve the health of multiple tissues, particularly, in the elderly, while minimizing the side effects associated with oncogenesis. The same combination may also be used to enhance OXTR expression in younger patients with a viral infection.

The inventors have found that unrelated viral infections in a subject may result in diminished levels of OXTR. In certain aspects, the amount of the OXTR agonist and ALK5 antagonist may be effective to treat a subject with a viral infection such that the OXTR expression in the subject are enhanced and are as compared to the OXTR expression from a healthy adult subject.

In some embodiments, the viral infection is caused by a virus of family Flaviviridae. In some embodiments, the virus of family Flaviviridae is selected from Yellow Fever Virus, West Nile virus, dengue fever virus, and Hepatitis C Virus. In other embodiments, the viral infection is caused by a virus of family Picornaviridae, e.g., poliovirus, rhinovirus, coxsackievirus, etc. In other embodiments, the viral infection is caused by a member of Orthomyxoviridae, e.g., an influenza virus. In other embodiments, the viral infection is caused by a member of Retroviridae, e.g., a lentivirus. In other embodiments, the viral infection is caused by a member of Paramyxoviridae, e.g., respiratory syncytial virus, a human parainfluenza virus, rubulavirus (e.g., mumps virus), measles virus, and human metapneumovirus. In other embodiments, the viral infection is caused by a member of Bunyaviridae, e.g., hantavirus. In other embodiments, the viral infection is caused by a member of Reoviridae, e.g., a rotavirus. In some embodiments, the virus is one that infects humans. In other embodiments, the virus is one that infects a non-human mammal, e.g., the virus is one that infects a mammalian livestock animal, e.g., a cow, a horse, a pig, a goat, a sheep, etc.

In certain aspects, the amount of the OXTR agonist and ALK5 antagonist may be effective to enhance hippocampal neurogenesis and reduce CD45+ cells in the brain of a subject (e.g. enhance neurogenesis and reduce brain inflammation), particularly an elderly subject. Without being bound to any particular theory, the inventors have found significantly more CD45+ cells in the old brains as compared to the young brains of mice. A 7 day treatment with an OXTR agonist and ALK5 antagonist was found to significantly reduce the CD45+ cells in the old brains of mice making the tissue more similar to that of young mice.

Alternatively or in addition, the amount of the OXTR agonist and ALK5 antagonist may be effective to enhance functional learning in a subject (e.g. improve memory and cognition).

In certain aspects, the amount of the OXTR agonist and ALK5 antagonist may be effective to attenuate p16 levels (i.e. reduce senescence) in multiple tissues (e.g. old muscles, liver, brain) and normalize the p16 levels in these tissues. In certain aspects, the amount of the OXTR agonist and ALK5 antagonist may be effective to reduce p16 levels in at least one tissue such that the p16 levels are as compared to the p16 of the same type of tissue from a younger subject.

Effective amounts of the OXTR agonist and/or ALK5 antagonist may readily be determined empirically from assays, from safety and escalation and dose range trials, individual clinician-patient relationships, as well as in vitro and in vivo assays such as those described in the art (e.g., Reagan-Shaw et al. (2007) The FASEB Journal 22:659-661).

The subject may be any suitable animal, such as a rodent (e.g., mouse, rat, etc.), primate (e.g., human, monkey, etc.), and so forth. In one embodiment, the subject may be a mouse. In certain embodiments, the subject may be a human. The subject may have a viral infection as well as, sarcopenia, muscle injury, cachexia, arthritis, an auto-immune disease, severe trauma, osteoporosis, obesity and related metabolic disorders, or any disease involving degeneration of tissue. In certain aspects, subject may be elderly, e.g., 60 years or older, 65 years or older, 70 years or older, 75 years or older, 80 years or older, 85 years or older, 90 years or older, and so forth. The subject may be a male or a female subject.

The OXTR agonist and ALK5 antagonist may be administered by any suitable route of administration, such as by enteric administration (e.g., oral) or by parenteral administration (e.g., intravenous, intra-arterial, intra-muscular, subcutaneous, etc.). For example, the OXTR agonist and ALK5 antagonist may be delivered by daily injections, nasal spray, using pump, delivered topically as a cream or using patches, oral tablets that prevent degradation of these bioactive molecules by the gastrointestinal tract. In addition, the OXTR agonist and/or ALK5 antagonist may be incorporated into a variety of formulations for therapeutic administration, according to any of the embodiments discussed herein. In certain cases, the OXTR agonist and ALK5 antagonist may be administered directly into a location in the body, such as, injured muscle, bone, a region of the brain (e.g., hippocampus), and the like.

In certain embodiments, the amount of an ALK5 antagonist to be administered may be gauged from the IC50 of the given ALK5 antagonist. By “IC50” is intended the concentration of an antagonist required to achieve 50% inhibition of a specific biological or biochemical function, such as ALK5 signaling.

With respect to the ALK5 antagonists of the present disclosure, an effective amount (e.g., the amount to be administered) may be 200× the calculated IC50 or less. For example, the amount (e.g., effective amount, amount to be administered, etc.) of an OXTR agonist and/or ALK5 antagonist may be 200× or less, 150× or less, 100× or less, 75× or less, 60× or less, 50× or less, 45× or less, 40× or less, 35× or less, 30× or less, 25× or less, 20× or less, 15× or less, 10× or less, 8× or less, 5× or less, 2× or less, 1× or less, 0.5× or less, or 0.25× or less than the calculated IC50. In one embodiment, the effective amount may be 1× to 100×, 2× to 40×, 5× to 30×, or 10× to 20× of the calculated IC50.

With respect to OXTR agonists and ALK5 antagonists of the present disclosure, the effective amount (amount to be administered) may be gauged from the EC50. By “EC50” is intended the plasma concentration required for obtaining 50% of a maximum biological effect. Suitable biological effects include binding of the OXTR agonist to OXTR, binding of the ALK5 antagonist to ALK5, OXTR agonist effect on OXTR signaling, ALK antagonist effect on ALK5 signaling, and/or downstream effects such as cell regeneration. An effective amount may be 200× the calculated EC50 or less. The amount (e.g., effective amount, amount to be administered, etc.) of an OXTR agonist and/or ALK5 antagonist may be 200× or less, 150× or less, 100× or less, 75× or less, 60× or less, 50× or less, 45× or less, 40× or less, 35× or less, 30× or less, 25× or less, 20× or less, 15× or less, 10× or less, 8× or less, 5× or less, 2× or less, 1× or less, 0.5× or less, or 0.25× or less than the calculated EC50. In one embodiment, the effective amount may be 1× to 100×, 2× to 40×, 5× to 30×, or 10× to 20× of the calculated EC50.

The OXTR agonist and ALK5 antagonist may exhibit a synergistic effect. As such, the effective amounts of the OXTR agonist and ALK5 antagonist may be less, e.g., half as much or less, than the effective amount of either agent alone.

Targeting and calibrating to healthy levels of distinct pathways (MAPK by OXTR agonist and TGF-beta by Alk5 antagonist) may promote a broad improvement in function of most mammalian cells, because MAPK and TGF-β signaling are the key cell-fate regulators.

In certain aspects, the OXTR agonist may be oxytocin administered by infusion or by local injection, e.g., by intravenous infusion at a rate of 0.01 μg/h to 100 μg/h, including 0.1 μg/h to 10 μg/h, 0.5 μg/h to 5 μg/h, etc. Administration (e.g., by infusion) can be repeated over a desired period, e.g., repeated over a period of 1 day to 5 days or once every several days, for example, five days, over 1 month, 2 months, etc. It also can be administered prior, at the time of, or after other therapeutic interventions. Alternatively, oxytocin may be administered by intramuscular injection, e.g., at an amount of 0.1 μg to 1000 μg, including 1 μg to 100 μg, 5 μg to 50 μg, 10μg to 30 μg etc.

In certain aspects, the effective amount of the OXTR agonist when administered in combination with the ALK5 antagonist may be 10% less, 20% less, 30% less, 40% less, 50% less, 60% less, or lesser than the amount of the OXTR agonist required to achieve the same effect when administered in absence of the ALK5 antagonist. In certain aspects, the effective amount of the ALK5 antagonist when administered in combination with the OXTR agonist may be 10% less, 20% less, 30% less, 40% less, 50% less, 60% less, or lesser than the amount of the ALK5 antagonist required to achieve the same effect when administered in absence of the OXTR agonist. Thus, the combined administration of the OXTR agonist and the ALK5 antagonist allows lowering the doses of each molecule, thus not skewing the respective pathways far from healthy signaling ranges, while maintaining and/or even broadening the positive effects on multiple tissues and their stem cells.

In certain aspects the combined administration of the OXTR agonist and the ALK5 antagonist may enhance expression of the OXTR by at least 10% more, 20% more, 30% more, 40% more, 50% more, 60% more, or more than the expression of OXTR by either molecule alone.

In certain aspects the combined administration of the OXTR agonist and the ALK5 antagonist may enhance OXTR expression by at least 10% more, 20% more, 30% more, 40% more, 50% more, 60% more, or more than the OXTR expression in the absence of the administration.

In certain cases, the effective concentration of the ALK5 antagonist (e.g., Galunisertib (LY2157299)) may be 0.05 μM-0.75 μM and the effective concentration of the OXTR agonist (e.g., oxytocin) may be 7.5 nM-30 nM for enhancing proliferation of myoblasts. In certain cases, the effective concentration of the ALK5 antagonist (e.g., Galunisertib (LY2157299)) may be 0.5 μM-3 μM and the effective concentration of the OXTR agonist (e.g., oxytocin) may be 10 nM-30 nM for enhancing proliferation of satellite cells of an old subject.

In certain aspects, the effective concentration of oxytocin may be in the range of 0.01 to 1 microgram per gram mouse body weight per day. In certain aspects, oxytocin may be administered by intraperitoneal or subcutaneous injection, or continually by osmotic pump. In certain aspects, the effective concentration of the ALK5 antagonist (e.g., 2-(3-(6-Methylpyridin-2-yl)-1H-pyrazol-4-yl)-1,5-naphthyridine) may be in the range of 2 to 200 picomoles per gram mouse body weight per day. In certain aspects, the ALK5 antagonist may be administered by intramuscular, intraperitoneal or subcutaneous injection, or continually by osmotic pump.

The effective concentrations of the OXTR agonist and ALK5 antagonist for a human subject may be extrapolated from effective concentrations derived from animal studies. For example, the following conversion table may be used for determining human equivalent dose:

TABLE 1
Conversion of Animal Doses to Human Equivalent
Doses Based on Body Surface Area

| | To Convert | To Convert Animal Dose in mg/kg | |
|---|---|---|---|
| | Animal Dose in | to HED^{a} in mg/kg, Either: | |
| | mg/kg to Dose in | Divide | Multiply |
| | mg/m², Multiply | Animal | Animal |
| Species | by kₘ | Dose By | Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula: HED = animal dose in mg/kg × (animal weight in kg/human weight in kg)^{0.33}.
^{b}This kₘ value is provided for reference only since healthy children will rarely be volunteers for phase 1 trials.
^{c}For example, cynomolgus, rhesus, and stumptail.

The treatment methods may also include an assessment of OXTR expression in the subject following administration with the OXTR agonist and the ALK5 antagonist. Based on the assessed expression or lack thereof, the amounts of the OXTR agonist and/or the ALK5 antagonist may be adjusted by increasing or decreasing the amounts and/or the schedule of administration of the OXTR agonist and the ALK5 antagonist.

In certain aspects, the method may include treating the subject with only one of the OXTR agonist and the ALK5 antagonist prior to or after treating the subject with both the OXTR agonist and the ALK5 antagonist.

### Utility

OXTR agonists may be used to treat and prevent sarcopenia, improve muscle regeneration after injury and prevent muscle mass loss observed after long term immobilization (bed rest or cast), as well as low gravity (space travel). Specifically, oxytocin is more desirable than other drugs on the market or under development, since it is physiologic, has virtually no side effects and is already FDA approved to induce labor in pregnant women and in clinical trials to treat children with autism. The plasmatic level of oxytocin decreases with age in mice. A short term subcutaneous injection (systemic delivery) of oxytocin is able to restore muscle regeneration in old mice and conversely, the injection of an oxytocin antagonist in young mice prematurely ages their muscle regeneration potential (see Elabd et al. (2014) Nature Communications 5:4082). Confirming the dependence of muscle maintenance and repair on oxytocin, mice with a knock out in oxytocin have defective muscle regeneration and premature sarcopenia (loss of muscle tissue). Oxytocin is also known to prevent osteoporosis and regulate fat distribution after menopause. Oxytocin inhibits p16 (marker and effector of senescence) in adult stem cells, thereby enabling their productive responses to maintain and repair tissues. The positive effects of oxytocin can be further supplemented by the small molecule inhibitor of TGF-beta receptor (ALK5 inhibitor), which simultaneously rejuvenates myogenesis and neurogenesis and reduces inflammation, when delivered systemically into 2-year old mice (equivalent of ˜85 year old human).

Summarily, systemic delivery of oxytocin and Alk5 inhibitor is capable of enhancing OXTR expression, combating and reversing the aging of multiple tissues, including muscle, brain and bone and is effective in down-modulating cellular senescence, reducing inflammation and reducing obesity (known to be associated with and exacerbate metabolic disorders). The mixture of these two molecules enables the long-term applications, in which each drug is not used at a high dose, therefore, the negative effects of down-regulation of TGFβ signaling and/or up-regulation of MAPK/pERK signaling are minimized, while the positive effects on health, maintenance and repair of multiple tissues and organs are maximized and/or optimal.

A combination of oxytocin (or another OXTR agonist) with Alk5 inhibitor may be used to enhance OXTR expression in a subject with a viral infection and potentiate the positive effects on muscle, bone, combat age-related fat deposition and to promote and rejuvenate hippocampal neurogenesis (leading to increase in memory and cognition and preventing loss of memory and cognition in the elderly).

### EXAMPLES

### Example 1

### Alk5i and Oxytocin Quickly Enhances Old Muscle Repair In Vivo and Avoids OXTR Down-Modulation

To develop a meaningful in vivo therapy we tested a dose curve of Alk5i and OT (alone and in combination) in freshly-isolated old muscle stem cells that were exposed overnight to their old serum, where proliferative capacity is typically inhibited. When used together, lower concentrations of Alk5i and OT suffice for enhancing the proliferation of these old muscle stem cells in old ex-vivo environment, as compared to each molecule alone.

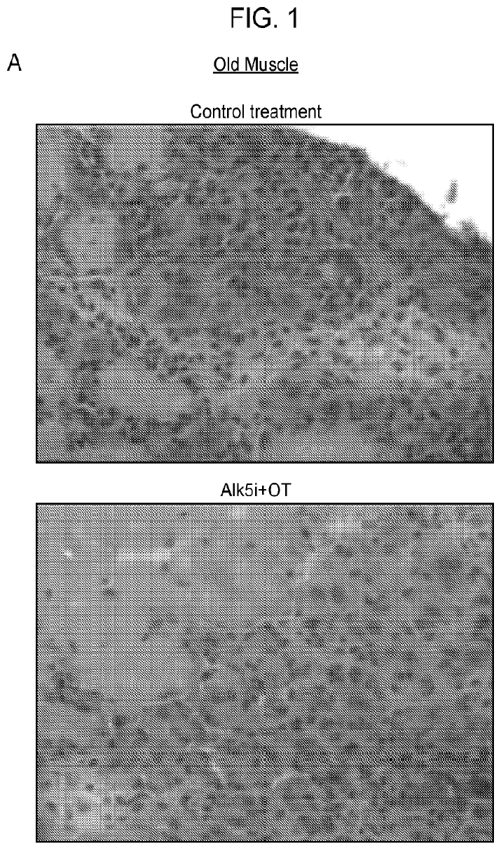

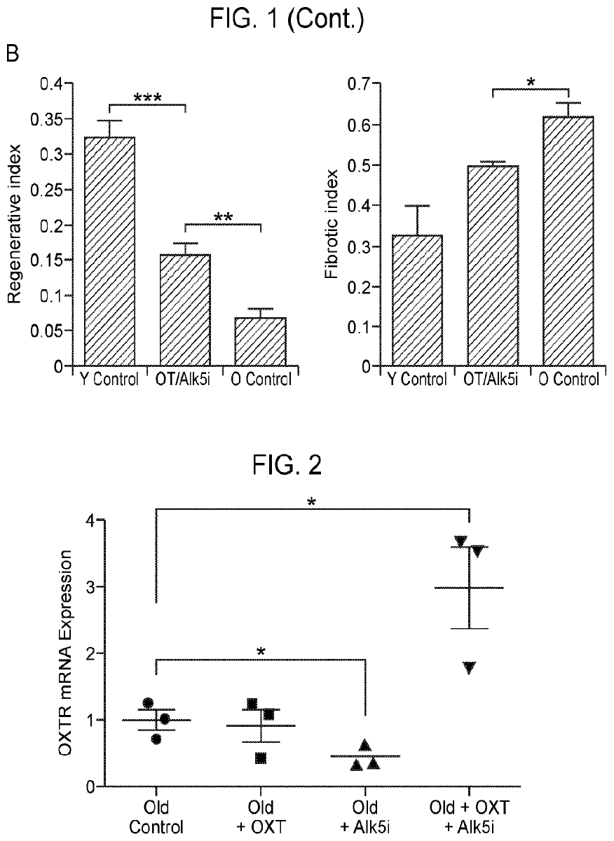

We then conducted in vivo studies where Alk5i was used at a 10-fold lower concentration than previously published in combination with OT. Old C57.B5 male mice (23-24 months of age) were treated with control vehicle (HBSS) or with Alk5i, OT or combined, for 2 days, after which TA and Gastroc muscle were injured with cardiotoxin (CTX). And after additional 5 days of control or OT and/or Alk5i injections, the success of muscle regeneration was determined based on the formation of de-novo myofibers (small, eMyHC+ with central nuclei) that effectively regenerate the injury site in young mice, but do not form well in the old muscle where fibrosis ensues. Young C57.B6 mice (2-3 months of age) injected with control vehicle (HBSS) served as a positive control for efficient muscle repair. The representative and quantified data shown in FIG. 1 demonstrate that Alk5i+ OT improved the regenerative capacity of old muscle, increasing the numbers of newly-formed muscle fibers and reducing fibrosis.

Old (23-24 mo) C57.B6 mice were injected subcutaneously with Alk5i+OT (0.02 nmol/g/day for ALk5i, and 1 ug/g/day for OT) or control vehicle (HBSS) for 2 days, daily; after which TA and Gastrocs were injured by CTX and the administration of Alk5i+ OT continued for additional 5 days. Young C57.B6 mice (3-4 mo) identically administered with HBSS for 7 days and injured with CTX were used as controls for effective muscle repair. A. TA muscles were sectioned to 10 micron and H&H staining was performed where newly formed muscle fibers are smaller and with central nuclei. These form efficiently in the young, but not old injured muscles. As shown in representative H&E panels, Alk5i+OT dramatically enhanced in vivo myogenesis: formation of dense rows of new myofibers and diminished fibrosis (white areas devoid of muscle fibers). B. Regenerative index and Fibrotic index were defined at 5 days post CTX injury, as in (21); Alk5i+OT improved muscle regeneration (*,**p<0.022), making fibrosis non statistically different in Old mice treated with Alk5i+OT as compared to Young mice.

### Example 2

### Alk5i and Oxytocin Avoids Alk5i-Promoted Down-Regulation of OXTR

In combination, an Alk5 antagonist and an OXTR agonist enhances OXTR expression. Interestingly, Alk5i reduced the levels of the oxytocin receptor (OTR) as compared to the control vehicle (HBSS) (FIG. 1). Alk5i and oxytocin (OXT) treatment resulted in a trend toward elevated levels of OXT in skeletal muscle of old animals, while OXT itself had no effect. Thus, combining Alk5i with OXT protects the OXTR from down-regulation, thereby avoiding the negative consequences of diminished OXT signaling that is needed for muscle maintenance and repair.

Old C57.B6 male mice (23-24 mo) per were administered by subcutaneous injections with oxytocin (OXT), Alk5i, a mixture of OXT and Alk5i (OXT+Alk5i) or control in vivo for 7 days, daily. The expression levels of oxytocin receptor (OXTR) were assayed by real-time qRT PCR in skeletal muscle of these mice. As compared to control HBSS, OXT did not have an effect on OXTR and Alk5i diminished OXTR levels by 2 fold (*p<0.05 Old Control compared to Old+Alk5i). OXT+Alk5i treatment prevented the OXTR down-regulation and enhanced the levels of OXTR expression in the muscle of old mice.

These results suggest that Alk5i+OXT might be more beneficial for skeletal muscle health than Alk5i alone, as it allows the maintenance of the pro-regenerative OXTR and enhances the repair of old muscle in a shorter time frame and at a much lower dose of Alk5i then previously reported.

### Example 3

### Hepatogenesis is Enhanced, and Liver Adiposity and Fibrosis are Reduced by Alk5i and Oxytocin

To examine whether Alk5i+OT is also effective in multiple tissues, mice were treated daily in vivo for 7 days with Alk5i+OT as described above, and the same animals that were examined for muscle repair after experimental injury were also assayed for hepatogenesis, measuring numbers of Ki67+albumin+cells), liver adiposity measuring staining by Oil Red O, and liver fibrosis, determining the numbers of albumin-clusters.

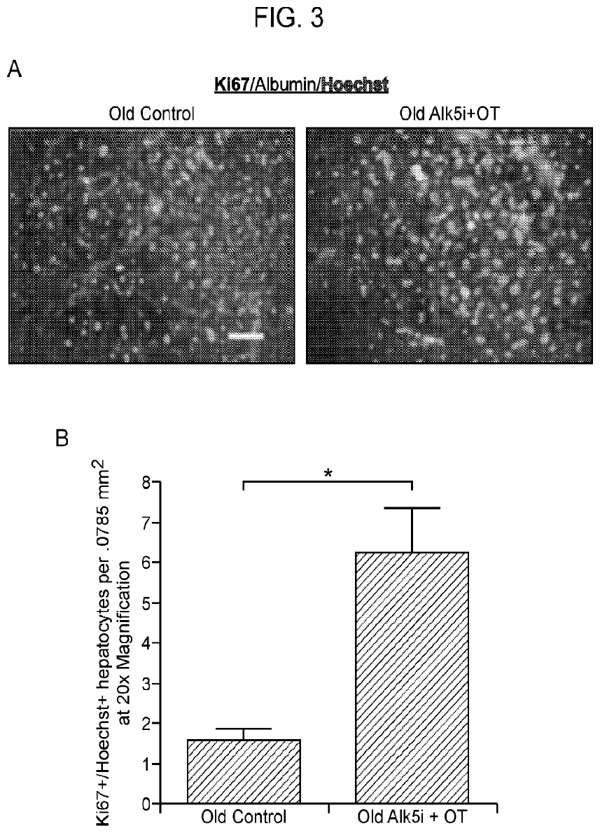

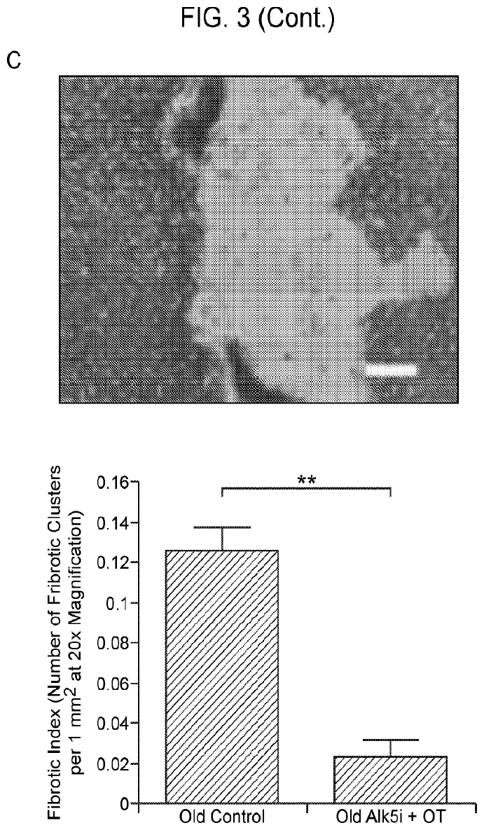

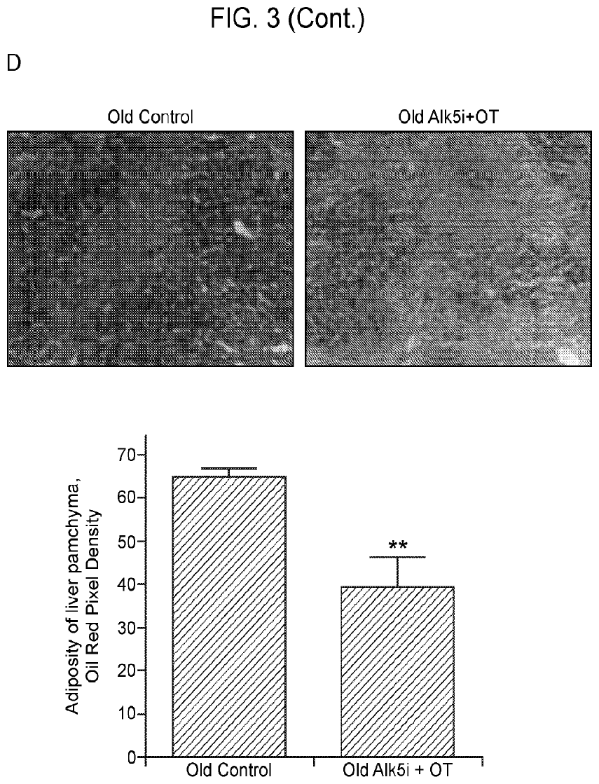

As shown in FIG. 3, the low dose of Alk5i combined with OT within one week enhanced hepatogenesis and reduced the adiposity and fibrosis of old livers, making these more similar to control young mice. Thus, to a similar extent reported for heterochronic parabiosis and blood apheresis, a defined pharmacology of Alk5i+OT exhibits positive effects not just on skeletal muscle, but also on liver health and maintenance in old mice.

Old (23-24 month) C57.B6 mice were injected subcutaneously with Alk5i+OT (0.02nmol/g/day for ALk5i, and 1 ug/g/day for OT) (O Alk5i+OT) or control vehicle (HBSS) (OC) for 2 days, daily; after which TA and Gastrocs were injured by CTX and the administration of Alk5i+OT continued for additional 5 days. Young C57.B6 mice (3-4 month) identically administered with HBSS for 7 days and injured with CTX were used as controls for efficient hepatogenesis and good liver health. A. 10 um liver sections were immunostained with Ki67+ (red), Albumin+(green) to indicate proliferating hepatocytes, Hoechst dye (blue) shows nuclei, representative images are shown. B, Quantification of Ki67+ hepatocytes per section showing an increase with Alk5i and OT. p<0.02. C. Albumin-ye fibrotic clusters of cells are more commonly found in old livers, and are quantified in D; the incidence of fibrosis is reduced by Alk5i+OT. p<0.05. E. Oil Red S staining of liver sections are quantified in F to show the decrease in liver adiposity with Alk5i+OT, as determined by Image J quantification of red pixel density, as published (21). p<0.05.

### Example 4

### Hippocampal Neurogenesis Improves in Old Injured and Non-Injured Mice That are Treated with Alk5i and Oxytocin

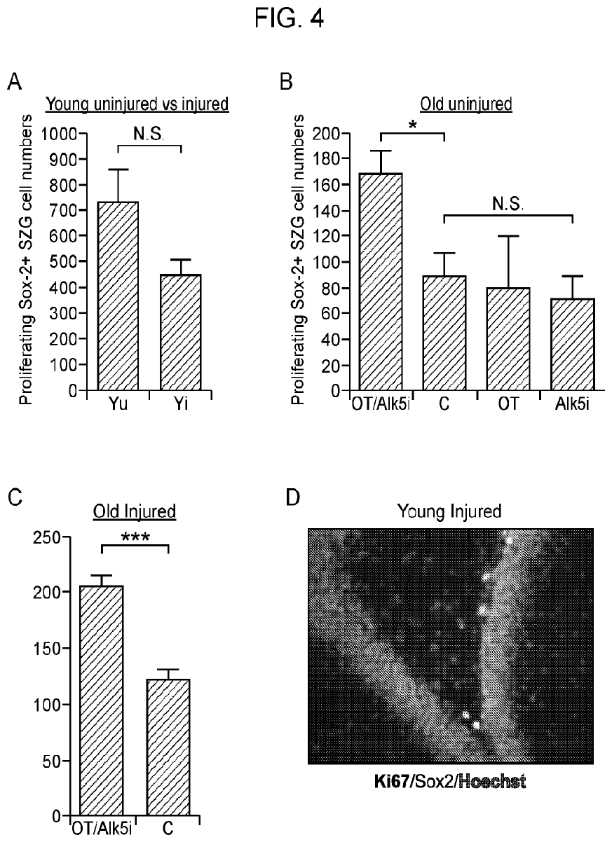

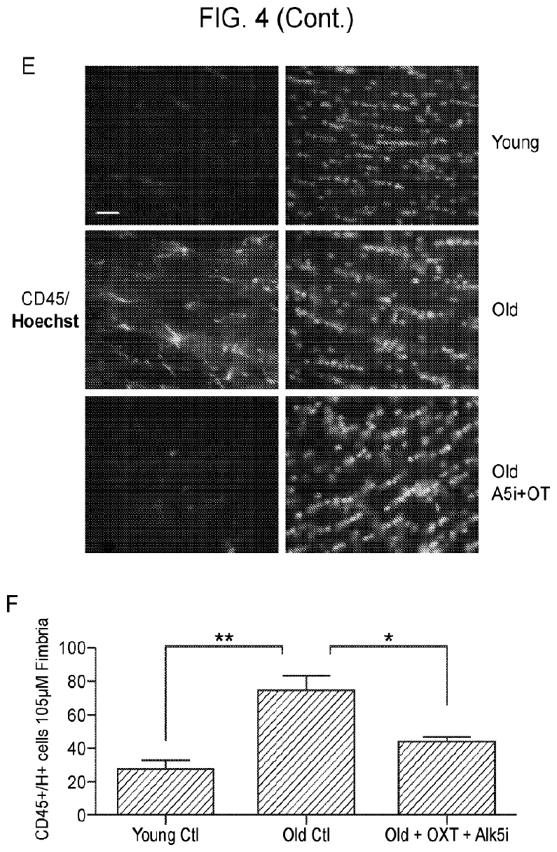

To determine whether Alk5i and oxytocin is capable of enhancing neurogenesis in the same old mice that showed improvement in muscle repair and liver regeneration and health, mouse brains were sectioned and analyzed for the numbers of proliferating (Ki67+ or BrdU+) neural stem cells (Sox-2+) in the subgranular zone (SGZ) of the Dentate Gyrus of hippocampus. In addition to the cohort that underwent muscle injury, we also performed a study with old mice that were not injured. Young mice administered with control HBSS served as control. Sox-2 expressing cells, some of which were also proliferating (e.g., Ki67+) were robustly identified in the SGZ location and both Sox-2 and Ki67 displayed nuclear immunofluorescence (FIG. 4D).

As shown in FIG. 4, Alk5i+OT in vivo treatment resulted in a nearly two-fold increase in hippocampal neurogenesis in just one week, in the same old mice that also manifested the enhanced myogenesis and improved liver health and regeneration. Further, young control uninjured mice have approximately 10 fold better SGZ neurogenesis than old. Peripheral muscle injury resulted in a non-statistical trend for diminished neurogenesis in the young animals. While still lower than young, neurogenesis in Alk5i+OT treated old animals became significantly improved with or without peripheral muscle injury (FIG. 4). Notably, OT alone or the low dose of Alk5i alone failed to enhance the neurogenesis of the old animals, which remained not statistically different than the HBSS control (FIG. 4C).

One negative causal factor in the old brain that contributes to neurogenic and neuroprotective decline is an age related change in microglia and central inflammation, orchestrated locally in brain, as well as an influx of peripheral leukocytes to the brain. To assess these phenotypes in our study, we quantified the numbers of CD45+ monocytic cells in brains of young and old mice, administered with HBSS control, and in brains of old animals that were treated with Alk5i+OT. As shown in FIG. 4E (quantified in 4F), significantly more CD45+ cells were detected in the old brains as compared to young, particularly at the fimbria of the hippocampal region. Interestingly, a 7-day treatment with Alk5i+OT significantly reduced these CD45+ cells that are indicative of a neuroinflammatory response in the brains of old mice, making the tissue more similar to that of young animals. These results demonstrate that Alk5i+OT quickly and robustly enhances SGZ neurogenesis, reduces brain inflammation and reduces CD45+ monocytes in old brains, simultaneously with improving muscle repair, hepatogenesis and liver health.

Hippocampal neurogenesis was assayed in the same mice that were studied for muscle repair above and additionally for mice that were not injured by cardiotoxin. The numbers of proliferating neural stem cells (Sox2+Ki67+) cells per DG of SGZ of hippocampus were quantified in serial brain cryosections.

With reference to FIG. 4A, as compared with the brains of uninjured mice (Yu), peripheral muscle injury resulted in the previously observed trend for diminished neurogenesis in young mice (Yi). NS=non-statistically significant. FIG. 4B shows old mice that were treated in vivo as indicated for 7 days, after which hippocampal neurogenesis was assayed by co-immunodetection of Sox-2 and BrdU (injected in vivo). Alk4i+OT but not OT alone or Alk5i alone significantly enhanced the numbers of proliferating Sox-2+ neural stem cells in the old hippocampi. *p<0.05. FIG. 4C, shows old mice that were treated in vivo as indicated for 2 days, after which muscle injury was performed with CTX and after 5 additional days of treatment with the OT, Alk5i, Alk5i+OT or HBSS, brains were isolated and studied for hippocampal neurogenesis via co-immunodetection of Sox2 and Ki67. ***p<0.05. FIG. 4D is a representative immunofluorescence staining of a brain section from HBSS-treated CTX-injured young animal is shown. FIG. 4E exhibits immunofluorescence on 35-micron brain sections derived from uninjured mice which was performed with anti-CD45 antibodies, using Hoechst to label all nuclei. A dramatic age-increase of CD45+ cells in old brains were observed. Scale bar=100 microns. FIG. 4F shows quantification of CD45+ cells in multiple serial sections of independent mice from each cohort demonstrated a significant reduction in old mice treated with Alk5i+OT, as compared to the old HBSS control animals. *,**p<0.05.

### Example 5

### Learning is Improved in Old Mice Treated with Alk5i and Oxytocin

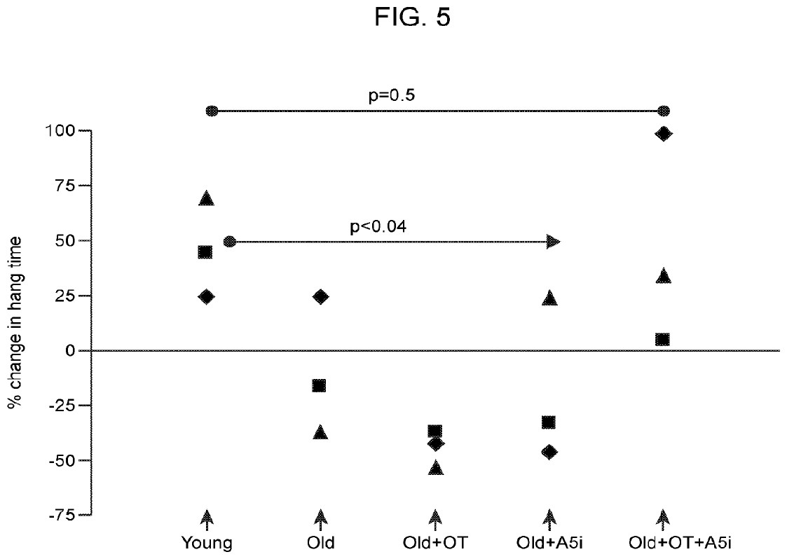

To assay functional performance, Alk5i alone (low dose), OT alone, Alk5i+OT and control HBSS were dosed to old mice without muscle injury, using young mice injected with HBSS as an additional control. The four-limb hanging test was given to all mice before and after the 7-day treatment. In order to better focus the test on the learning and memory aspects (rather than strength) we examined the percent improvement of each individual animal from the first to the second test session among all studied cohorts. As expected, the young animals significantly improved while the old (control) did not. Old animals treated with Alk5i or OT alone did not show improvement and were similar to the old control and statistically different from the young control (p<0.04). Very interestingly, in vivo treatment with Alk5i+OT showed a strong improvement trend over old control (p=0.09), making this cohort to improve significantly similar to young (p=0.5, test, 1 tail, heteroscedastic) (FIG. 5). At the same time, when the data analysis was focused on animal strength (the average hang time) instead of learning (percent improvement from first to second hang time), young animals outperformed all old cohorts, regardless of whether HBSS, Alk5i, OT or Alk5i+OT were administered. This suggests that Alk5i+OT in one week improves the learning and memory of old animals, being more therapeutic than blood exchange. These results also suggest that old animals that were administered with Alk5i+OT did not significantly gain strength in the 7-days between the first and second sessions, but specifically improved their learning and memory on how to perform in this test.

A four-limb hanging test was conducted with mice before and after treatment by injection of oxytocin (OT), Alk5 inhibitor (A5i) or both, or control HBSS (young and old). The maximal hang time from two trials was considered and the percent change for each animal before and after treatment is shown. A difference is observed between young and all old cohorts (p<0.04) except for the old+Alk5i+OT where there is no statistically significant difference with the young (p=0.5) (test, 1 tail, heteroscedastic). Thus, Alk5i+OT improves learning of old animals, while OT alone or Alk5i alone do not.

### Example 6

### Alk5i and Oxytocin Attenuate p16 Levels in Multiple Tissues of Old Mice In Vivo

The levels of p16 and other CDK inhibitors, p15, p21 and p27, increase with age in muscle stem cells, interfering with productive regeneration. OT has been shown to down-modulate CDKI p21 in a pERK-dependent manner, but the effects on p16 were not studied. Similarly, at a high dose, Alk5i attenuates p21 levels in brain of old mice, but the data on p16 was lacking.

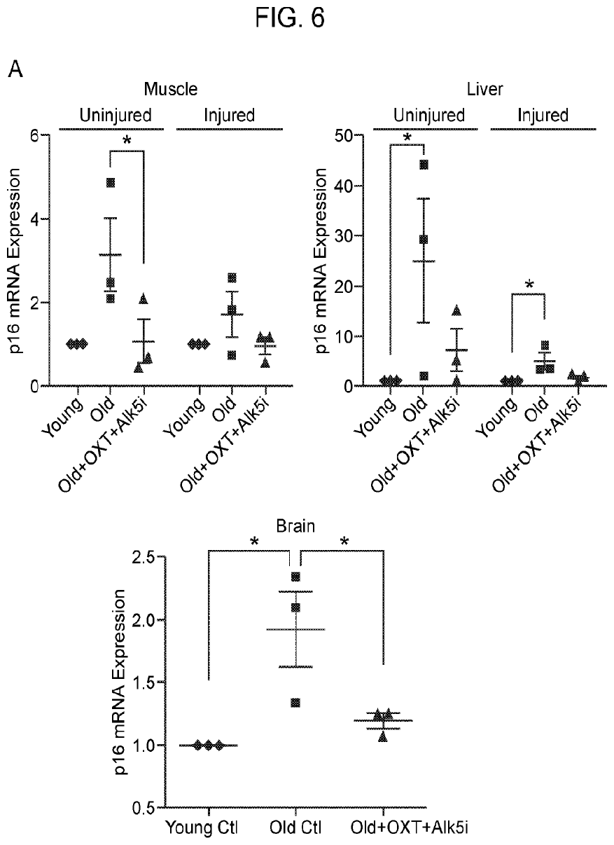

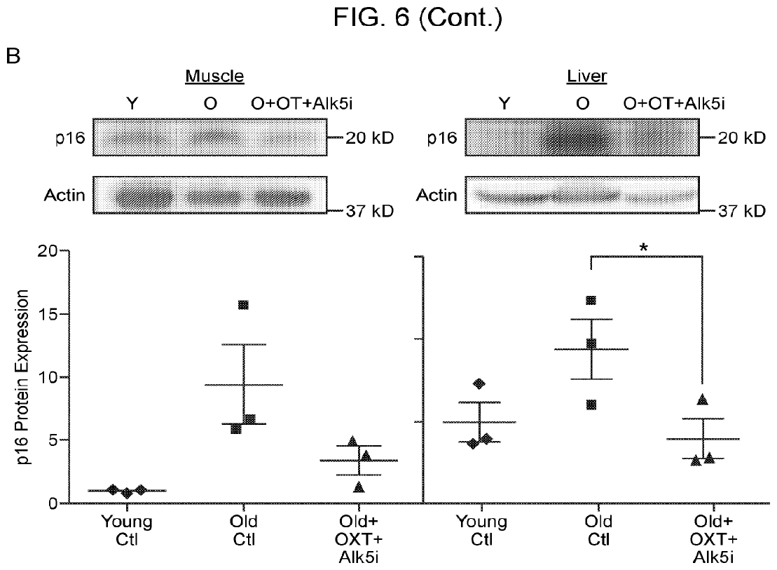

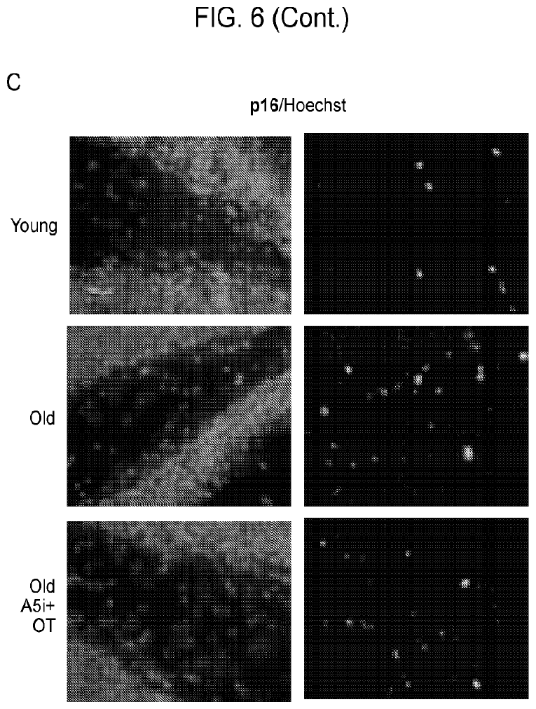

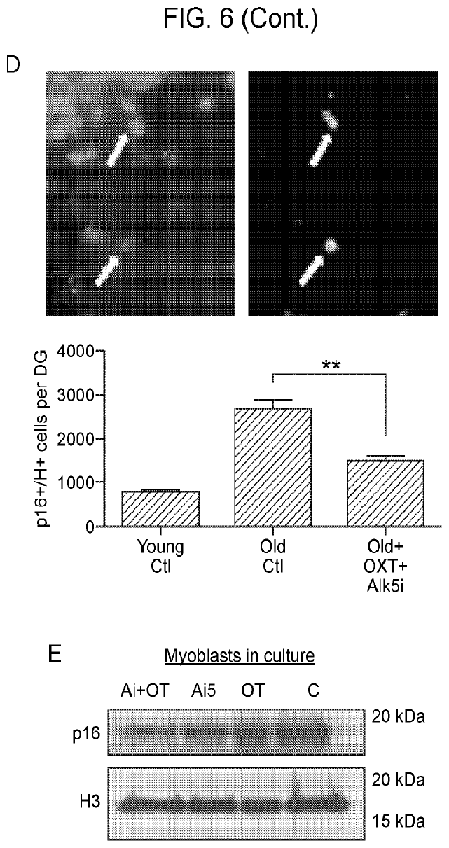

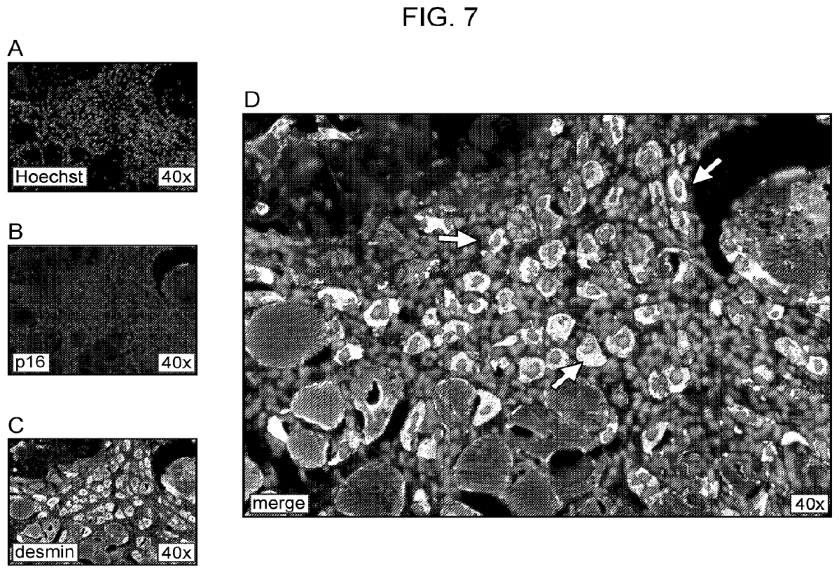

The effects of in vivo administered Alk5i+OT on the p16 levels in muscle, liver and brain were studied. It was observed that p16 becomes elevated with age, and that muscle injury attenuated p16 in old muscle, consistent with the higher cell proliferation required when muscle regenerates after injury by CTX, and interestingly p16 was also attenuated in liver, consistent with the increased cell proliferation seen there (FIG. 6 A). Low dose of Alk5i combined with OT in only 7 days normalized the levels of p16 in all of the old tissues studied, making them similar to those of young mice (FIG. 6 A-C). Additionally, as compared to young mice, a high variation in p16 mRNA levels was observed in old mice and Alk5i+OT made p16 expression range tighter and more similar to that of young animals. While for some cohorts only a trend toward attenuation was observed at the mRNA levels (for example, liver from uninjured mice), such down-regulation became statistically significant at protein levels (FIG. 6A, B). For muscle, a trend of attenuation of p16 protein reached statistical significance at the mRNA levels (FIG. 6A, B). For the brain, p16 was significantly attenuated by Alk5i+OT at both mRNA and protein levels (FIG. 6A and C). Taken together, Alk5i+OT resulted in statistically significant down-modulation of p16 expression at the levels of mRNA, protein or both, in all studied tissues. Finally, in a proof of rapid and direct effect, Alk5i+OT, but not either molecule alone, down-regulated p16 protein in primary myotubes in 24 hours. In support of the notion that at some levels p16 is needed for normal adult myogenesis, we found many p16-high nuclei in young muscle regenerating after an injury, and in de-novo formed myofibers, the terminal myogenic differentiation cell-fate (FIG. 7). These results establish that p16 becomes elevated with age in muscle, liver and brain and the low dose of Alk5i combined with OT quickly normalizes p16 in all studied tissues in vivo, making them more similar to those of young mice.

Old male C57.B6 mice (23-24 mo) were injected subcutaneously with Alk5i plus oxytocin (Alk5i+OXT), or control vehicle (HBSS) for 7 days, daily. 3 month C57.B6 male mice identically injected with HBSS were used as young controls. Some animals were injured by CTX in their muscle, while others were uninjured. With reference to FIG. 6A, levels were determined in skeletal muscle, liver (from mice injured and not) and brain (from injured mice) by real-time qRT PCR. Alk5i+OT attenuated p16 mRNA levels making its levels closer to those of young mice; and muscle injury reduced p16 mRNA in muscle and liver. *p<0.05. With reference to FIG. 6B, Western blotting was performed on muscle and liver from uninjured animals. Representative images and quantification—scatter plots of independent in vivo experiments performed with each cohort are shown. Alk5i+OT significantly diminished p16 protein levels in these tissues. *p<0.05. FIG. 6C shows brain sections from uninjured mice were immunostained for p16 (red); Hoechst (blue) labels nuclei. Shown are images of hippocampal Dentate Gyri with visibly more p16 high cells in old brains as compared to young; Alk5i+OT reduced the numbers of p16 high cells in old brains. Scale bar=100 micron. A larger resolution image shows nuclear localization of p16 (arrows point to the same cells on p16-red and Hoechst-blue fluorescence). With reference to FIG. 6D, quantification of p16+cell numbers in serial sections from multiple animals of each cohort demonstrated that in vivo Alk5i+OT treatment significantly reduced the numbers of p16+cells in old brains (fimbria of hippocampus). Westerns on brain were precluded by perimortem perfusion—fixation. E. To examine a direct effect on p16 levels, primary myoblasts were differentiated for 5 days in DMEM, 2% horse serum, after which primary myotubes were treated with Alk5i, OT, Alk5i+OT or left untreated for 24 hours. Western Blotting with anti-p16 and anti-Histone H3 antibodies was performed. Alk5i+OT, but not either molecule alone, down-regulated p16 protein levels in primary myotubes in 24 hours.

### Materials and Methods Examples 1-6

Animals: Young male C57BL/6 mice were purchased from the Jackson Laboratory (#00664). Old male C57BL/6 mice (20-24 month) were purchased from the National Institute on Ageing.

Oxytocin (OT) was purchased from Bachem (H-2510) and a 30 mM stock prepared in sterile water.

Alk5 inhibitor (A5i), TGF-β1 Type I Receptor Kinase Alk5 inhibitor 2-(3-(6-Methylpyridin-2-yl)-1H-pyrazol-4-yl)-1,5-naphthyridine, was purchased from Enzo Life Sciences, and a 25 mM concentrated stock dissolved in DMSO.

OT and/or A5i or control vehicle (HBSS) was injected subcutaneously to old male C57.B6 mice (23-24 month), daily for 7 days before sacrifice. 3 month C57.B6 male mice identically injected with HBSS were used as young controls

Cardiotoxin muscle injury: two days after the start of oxytocin and/or Alk5 inhibitor or control, mice were injured by intramuscular injections of cardiotoxin (Sigma, 10 ul per muscle at 0.1 ug/ml) into the tibialis anterior (TA). Five days after the injury, TA muscles were isolated.

Four limb hanging test: Muscle strength and endurance were determined by timing how long mice can hang upside down from a wire screen, as published (27). Briefly, each mouse was placed on a 1 cm. mesh, 1 mm wire screen, 30 cm over soft bedding, inverted and timed until the mouse fell. Each animal was given two to three trials with at least a 5-minute rest between trials. Hanging index is expressed as the maximum hang time times the weight of the mouse, with longer times or greater weight for a given time considered better performance.

Antibodies were used at 0.5-1 ug/ml:
Albumin R&D Systems mouse MAB1455, 1:1000
Beta-actin: Thermo Scientific MA5-15739
Histone H3: Abcam ab8580
Ki67: Abcam rabbit ab16667, 1:200
p16: Abcam 189034
Sox2: Santa Cruz Biotechnologies sc-17320, 1:400
Isotype-matched IgG were used as negative controls (Sigma Aldrich)
goat anti-rabbit Alexa 546: Invitrogen A11010, 1:2,000
goat anti-mouse alexa 488: Invitrogen A11029, 1:2,000
bovine anti-rabbit IgG-HRP sc-2370
DNA was stained by Hoechst DNA dye at 1 μg/ml: Hoechst 33342 from Sigma Aldrich (B2261).

Tissue isolation was performed postmortem. Brains from uninjured animals were harvested and incubated in 4% paraformaldehyde (PEA) overnight at 4° C. and subsequently stored in 30% sucrose. Brain, liver, and muscle harvested from injured animals and muscle and liver from uninjured animals were snap-frozen in isopentane (−70° C.) and embedded in Tissue-Tek Optimal Cutting Temperature (OCT, Sakura Finetek, The Netherlands).

Tissue sectioning, Hematoxylin and Eosin staining, immunofluorescence of brain, muscle, and liver sections directly collected on positively charged frosted glass slides. Brain, liver, and muscle sections were 25 μm, 10 μm, and 10 μm respectively. Tissues were fixed with 70% ETOH, permeabilized with TitonX for 10-15 min on ice, incubated with primary antibodies at 4 C overnight in PBS+1% BGS, washed in this buffer and incubated with secondary fluorochorme-tagged antibodies and Hoechst for 2 hours at RT. IgG controls with isotype-matched antibodies were routinely done and non-specific fluorescence was minimal.

Free-floating immunofluorescence was performed on 35 μm thick brain sections obtained from the uninjured animals. Brains were frozen and sectioned with a freezing microtome. Sections were stored at −20° C. in cryoprotective medium comprised of 30% glycerin, 30% ethylene glycol, and 40% 0.05 M sodium phosphate buffer. Sections were washed with 1× Tris-buffered saline and 0.05% tween-20 (TBST), permeabilized with 0.1% triton-×100 detergent (in TBST) for 20 minutes and washed again, blocked with 10% bovine growth serum (BGS) in TBST for 1 hour and incubated with primary antibodies for p16 and CD45 at 1 ug/ml in TBST-BGS overnight at 4° C. Secondary antibodies and Hoechst DNA dye were used at 1 ug/ml for 2 hours at room temperature.

Oil Red Staining: 10 micron liver sections were hydrated in 1× PBS for approximately 10 minutes. The sections were then washed in 60% isopropanol for 3-5 minutes and later placed in isopropanol-based Oil Red 0 staining solution for 15 minutes. After, the sections were washed in 60% isopropanol once more for 1 minute. Nuclei on these sections were stained by a 5-minute wash in hematoxylin. The sections were finally washed in deionized water for one minute. Fluoromount was used as the mounting medium and images were taken from these slides.

Western Blot Analysis: Tissue lysates from frozen tissues embedded in OCT washed in 50% methanol to dissolve OCT, sedimented by centrifugation at 5,000 g for 5 minutes and solublized in Laemmli buffer. Proteins from brain tissue embedded in OCT were extracted with T-PER Tissue Protein Extraction Reagent (ThermoFisher Scientific #78510), using ¼ of the amount suggested by the manufacturer. Primary cell lysates were prepared by collecting cells in Laemmli buffer. All samples were brought to Laemmli buffer and heated at 95 C for 5 minutes before resolving by SDS-PAGE on precast 4-20% or 10-20% TGX gels (Bio-Rad), and transferred to 0.2 μm polyvinylidene difluoride membranes (Millipore). Western blots were blocked with 5% non-fat-dry-milk in phosphate buffered saline/0.05% Tween-20 (PBST) for 2 hours at room temperature, incubated with primary antibodies (p16: 1:2000, actin: 1:1000) at 4 C overnight, washed in PBST and incubated with secondary antibody for 2-4 hours. ECL was performed using Western Bright ECL reagent (Avansta), imaged on a Bio-Rad Gel Doc/Chemi Doc Imaging System with Quantity One software, and quantifications were carried out using ImageJ (NIH).

qPCR analysis: Tissue lysates from frozen muscle, liver, and brain embedded in OCT were prepared. 40 slices of 10 μm thick slices were collected and homogenized using QIAShredder (from QIAGEN). Total RNA was extracted with RNeasy Mini Kit (from QIAGEN) according to manufacturer's instructions. Reverse transcription was performed with Superscript III First-Stand Synthesis System (from Invitrogen) according to manufacturer's instructions. For real-time PCR amplification and quantification of gene of interest, 1 ug of total cDNA was used for the initial amplification using specific primers to each gene of interest; amplification was performed with a denaturation step at 95 C for 10 minutes, followed by 40 cycles of denaturation at 95 C for 15 seconds and primer extension at 60 C for 30 s. Real-time PCR was performed using iQ™ SYBR Green Supermix (from Bio-Rad) under CFX Connect Real-Time System (Bio-Rad). Reactions were run in triplicates. Housekeeping gene actin was used as an internal control to normalize the variability in expression levels, and results were analyzed using the 2-ΔΔCT method described (50).

Primers:

| (SEQ ID NO: 04) | |
|---|---|
| p16-f: | CGTACCCCGATTCAGGTGATG |
| (SEQ ID NO: 05) | |
| p16-f: | GCCGGATTTAGCTCTGCTCT |
| (SEQ ID NO: 06) | |
| actin-f: | CGCCACCAGTTCGCCATGGA |
| (SEQ ID NO: 07) | |
| actin-r: | TACAGCCCGGGGAGCATCGT |
| (SEQ ID NO: 08) | |
| OXTR-f: | GATGTCGCTCGACCGCTG |
| (SEQ ID NO: 09) | |
| OXTR-r: | CGGTACAATGTAGACGGCGA |

Data quantification: Muscle regeneration indices were calculated by counting percent de-novo myofibers with central nuclei to total nuclei in multiple images of 10-micron muscle sections for independently treated animals. Muscle fibrosis was quantified by measuring fibrotic areas on Image J. These fibrotic areas were normalized to the area of the image taken at 20× (˜14000 micron\hat{ }2). Hippocampal neurogenesis (Sox2+/Ki67+, Sox2/BrdU+), p16+SGZ cell numbers and CD45+ cell numbers were quantified by counting the number of respective cells (from multiple 25-35-micron IF brain sections) and for neurogenesis and p16+ cells, extrapolating these numbers to the total thickness of the dentate gyrus. Counting cell numbers from multiple 10-micron sections for each cohort produced the quantification of hepatocyte proliferation, e.g., Ki67+, albumin+, and Hoechst+ cells and of albumin-Ki67+ fibrotic cells. Oil Red O quantification was done in 10-micron liver sections, by obtaining the total area of the red fatty droplets as a percentage of the entire 20× image area, using a consistent color-threshold function in software ImageJ. At least three independently treated animals per each cohort (young control, old control, old+Alk5i, old+0T, old+Alk5i+OT) were used in quantification for each tissue and assay; and such assays (qRT PCR, IF, Western Blotting, functional performance test) were performed in 3-4 replicates. Student t-test and/or ANOVA were performed and p values of 0.05 or less were considered statistically significant.

### Example 7

### Control Lentiviral Vectors Down-Regulates OXTR in Mouse Myogenic Cells In Vitro and Mouse Muscle Stem Cells In Vivo

Lentiviral-mediated RNAi and transgenic gene expression have proven to be highly efficient and typically use empty vector backbones and non-target shRNA as controls. However, the influences of the vector controls on genes other than targeted were not well studied.

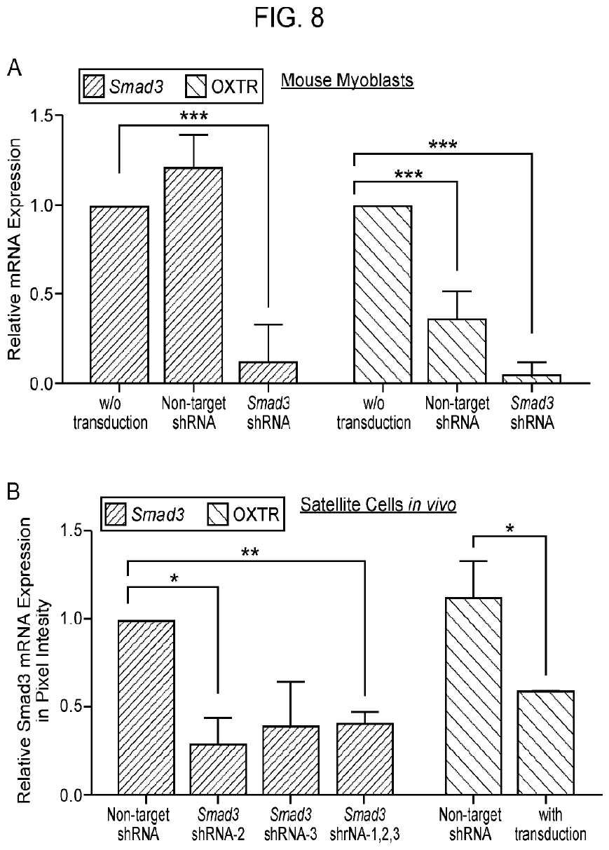

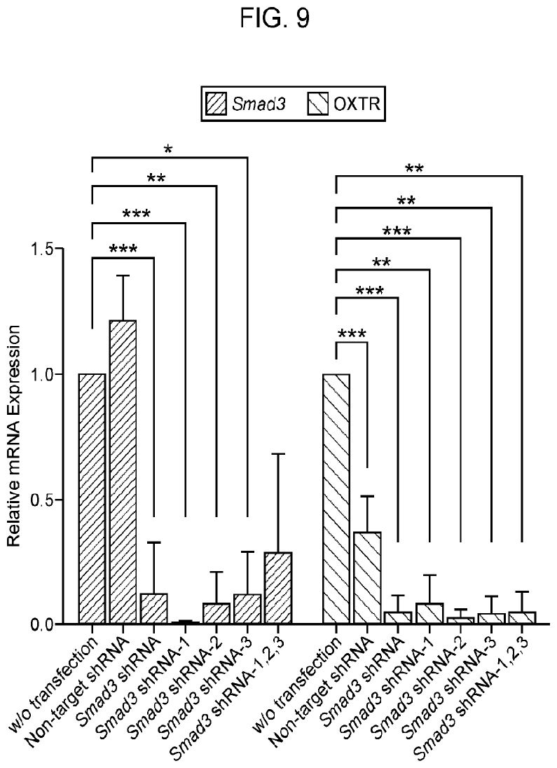

Primary mouse myoblasts were transduced with Smad3-targeting shRNA and non-mammalian, non-target shRNA controls, and compared the expression levels of Smad3 and OXTR with those of primary mouse myoblasts receiving a mock transduction of no viral vectors. As expected, the non-mammalian shRNA control served as a reliable reference for the down-regulation of Smad3 mRNA by the Smad3 targeting shRNA (FIG. 8a). However interestingly, the non-target shRNA vectors significantly down regulated OXTR expression by ˜70%, as compared to the un-transduced primary mouse myoblasts (FIG. 8a). Furthermore, the down-regulation of Smad3 by the Smad3-targeted shRNAs further diminished OXTR expression, as compared to non-target shRNAs (FIG. 8a, FIG. 9).

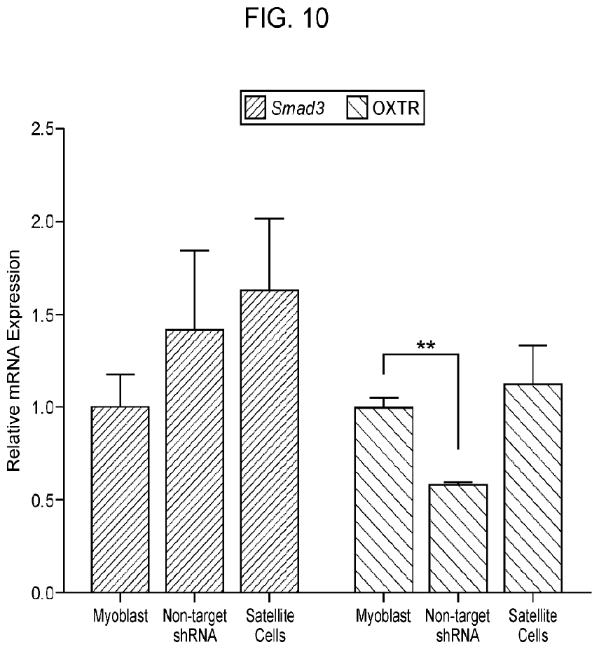

To examine if a similar phenomenon occurs in vivo, old C57.B6 mice, in which TGF-beta/pSmad3 pathway becomes elevated with age, were injected intramuscularly with non-target non-mammalian (GFP) shRNA viral particles, versus three different Smad3-targeting shRNAs. The transduced muscle satellite cells were isolated from the in vivo injected muscles 3 days later. The in vivo transduced satellite cells showed the expected down-regulation of Smad3 when muscle was injected with Smad3-targeting shRNA viral particles, as compared to the non-target shRNA vector (FIG. 8b). Very interestingly, the in vitro observed trend of OXTR down regulation by viral transduction was also detected in these in vivo studies (FIG. 8b, FIG. 10). Similar to the in vitro results, Smad3 shRNA in vivo additionally down-regulated OXTR, as compared to the non-target shRNA viral vector (FIG. 8b).

While non-mammalian control shRNA vectors are designed to not target any known, canonical mammalian sequences, the presence of shRNA will engage with and activate the RNA-induced silencing complex (RISC). Interested in whether the down-regulation of OXTR comes from viral transduction or RISC activation, primary mouse myoblasts were transduced with an empty vector (encoding just the viral backbone) that does not contain a hairpin insert, or with the above non-mammalian (GFP) shRNA control particles. OXTR expression levels were compared with those of non-transduced primary myoblasts. Data shows that both control vectors caused a significant (˜70%) downregulation in OXTR expression even at MOI=0.05, which is much lower than the titer that is typically recommended for viral transductions; and that control shRNA particles down-regulated OXTR faster than the viral backbone particles (FIG. 11A, B).

These data establish that transduction with control viral vectors perturbs key cell signaling molecules, such as OXTR at low viral titers.

### Example 8

### OXTR Downregulation Upon Lentiviral Transduction is Evolutionarily Conserved Between Mice and Humans, and Manifests Upon Different Viral Infections in Human Populations

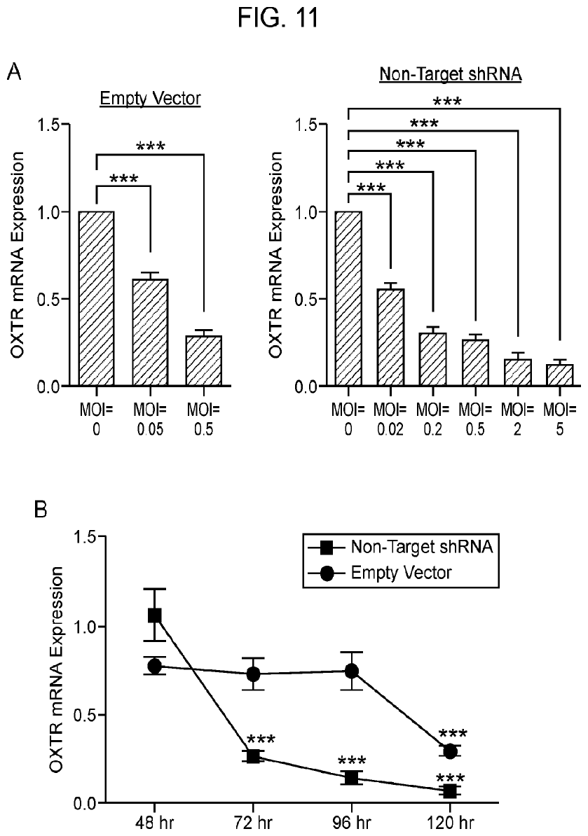

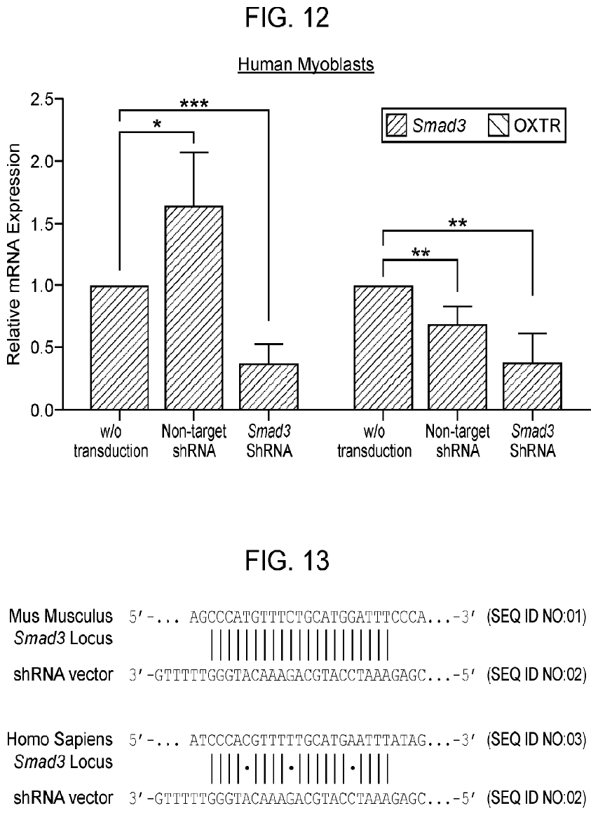

Interested in exploring the conservation of the observed phenomena between mouse and human cells, we repeated in vitro experiments from Example 8 with primary human myoblasts. Transduction of these human muscle precursors with non-mammalian, non-target (GFP) shRNA controls showed significant down regulation in OXTR expression, suggesting that OXTR downregulation upon lentiviral transduction is evolutionarily conserved between mouse and human. And similar to the outcomes with mouse myoblasts, this attenuation became stronger when a Smad3-targeting shRNA vector was used (e.g. when human Smad3 expression was experimentally diminished (FIG. 12). An examination of the complementarity demonstrated that mouse and human Smad3 loci have very high homology in the area targeted by this specific shRNA (FIG. 13) consistently with our data that this specific Smad3 shRNA targets Smad3 in both species.

Extrapolating these data, and to confirm that OXTR down-regulation would generally be caused by viral infections in humans, published GEO databases from human studies that focused on different viral infections (HIV, SIV, influenza virus, etc.) was analyzed. Specifically, previously published GEO databases were data mined that report the levels of OXTR, Smad3, TGFBR1, and TGFBR2, which were analyzed with statistical rigor in the presence of various viral infections (in various cell types), as compared to the non-infected control groups within each human study.

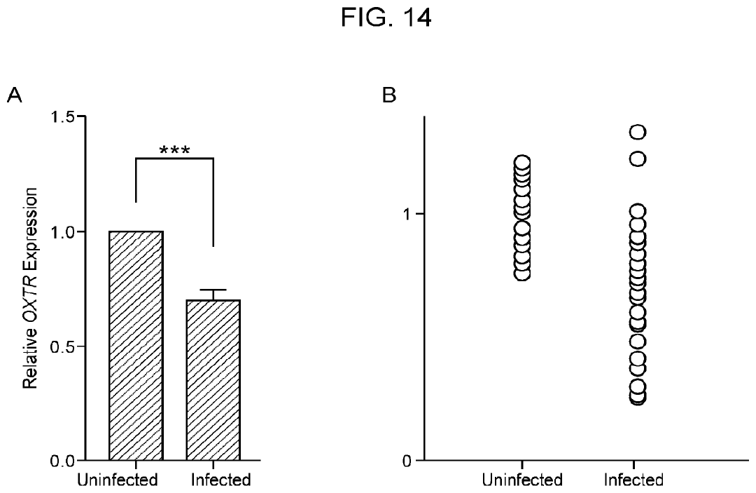

Interestingly, this analysis confirmed and extrapolated our conclusions by revealing that OXTR becomes significantly down-regulated upon different unrelated viral infections in human populations (FIG. 14a). In addition to pooling the results from all databases, the data from each individual database were also examined: for each group, OXTR showed down-regulation upon viral infections ranging from 47% to 82%, as compared with the level of this receptor in healthy individuals (FIG. 14b).

These results demonstrate an evolutionary conservation of OXTR attenuation by experimental and natural viral infections and indicate a significance of the uncovered phenomenon for human health.

### Example 9

### Lentiviral Transductions Do Not Down-Regulate Cell Surface Receptors Broadly

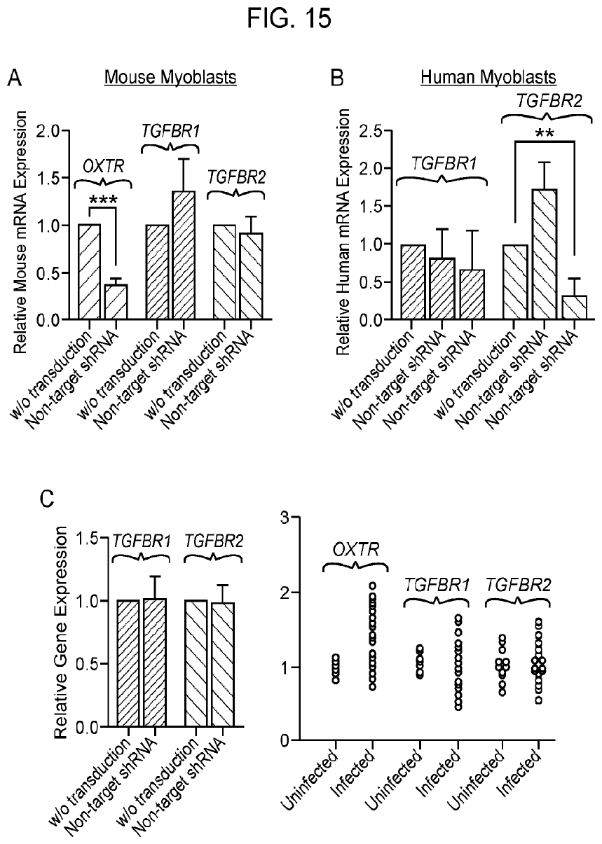

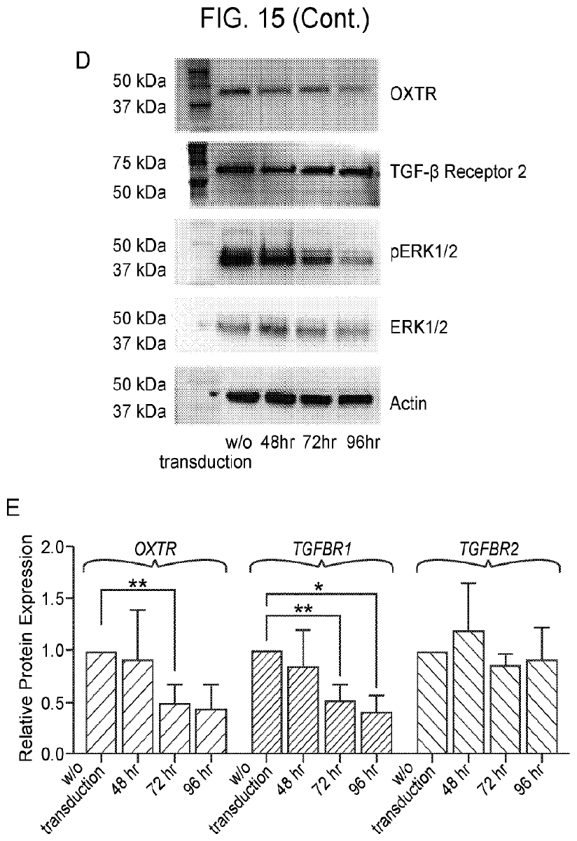

To answer whether the down regulation of receptors by non-target shRNA vectors applies to only certain proteins exemplified by OXTR, or manifests more generally, we examined the levels of TGFβ receptors 1 and 2 (TGFBR1, TGFBR2) in primary mouse and human myoblasts by real-time qRT-PCR and Western Blotting. Interestingly, in contrast to the OXTR, expression levels of TGFBR1 & 2 were not significantly altered upon control lentivirial transduction in either mouse or human myogenic cells, and TGFBR2 actually became elevated in human myoblasts (FIG. 15a, b).

Moreover, while OXTR was significantly down-regulated (FIG. 14), the expression levels of TGFBR1, TGFBR2, and Smad3 genes were not diminished in people afflicted with viral infections, as per our data mining study with the above-described human GEO databases (FIG. 15c). As for Smad3 expression, some human studies showed an obvious up regulation in expression upon viral infection, while others showed no change from healthy controls (FIG. 15c).

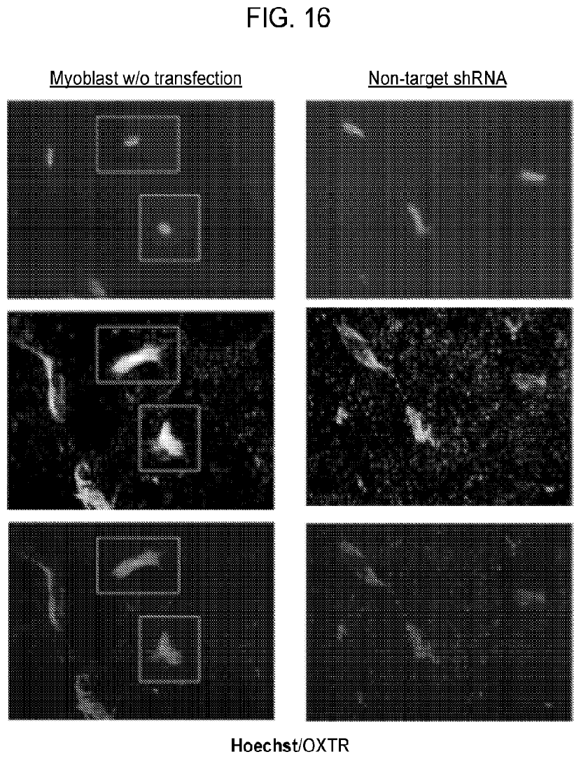

The expression levels of OXTR were further examined at protein levels through Western Blotting and were found to be reduced after the control lentiviral transduction, in a time-dependent manner (FIG. 15d). This downregulation of OXTR was further verified by immunofluorescence (FIG. 16). Furthermore, a key signaling molecule down-stream of OXTR, pERK, became attenuated concordantly with OXTR and with the same timing (FIG. 15d). In contrast, the protein levels of TGFBR2 did not change significantly, in agreement with our qRT-PCR results.

These results establish that viral infection promotes selected downregulation of certain, but not all cell surface receptors, which may perturb signaling networks. More specifically, these data suggest a skewing from OXTR/pERK to TGF-β/pSmad3 signaling upon viral infections, which is reminiscent of the age-imposed changes that associate with broad degenerative pathologies. Interestingly, in human populations such conserved pathway skewing took place regardless of the specific viral infections or their distinct mechanisms of action or diseases.

### Example 10

### Viral Infections Decrease Myogenic Activity In Vitro

Since OXTR signaling plays a major role in maintaining myogenic activity and muscle health, without being bound to any particular theory, it was hypothesized that control viral transductions, which attenuate OXTR, would also diminish the myogenicity of even young muscle progenitor cells.

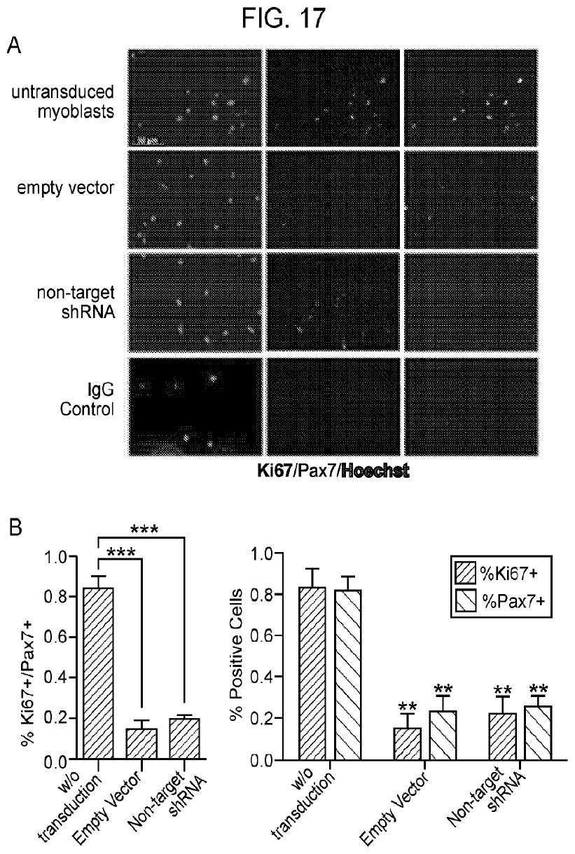

To examine this hypothesis, the in vitro proliferation of myogenic (Pax7+) cells that were derived from young mice were compared. Specifically, primary mouse myoblasts were transduced with either empty vector (encoding just the viral backbone) or with non-mammalian (GFP-encoding) shRNA particles, and were compared with the mock-transduced cohort. All cells were fixed at 5 days after the transduction and were analyzed by co-immunodetection of Pax7+ and the proliferation marker Ki67 (FIG. 17a). The percentage of Ki67+/Pax7+ double positive, as well as Ki67+ and Pax7+ single positive myoblasts out of all Hoechst+ cells were quantified and compared between the cohorts (FIG. 17b).

Interestingly and in complete agreement with our above observations, as compared to the un-transduced myoblasts, transduction with either the viral backbone or with the control shRNA particles dramatically and with high statistical significance reduced the percent of proliferating primary muscle progenitor cells from ˜85% down to ˜20%. Additionally, the numbers of cells expressing Pax7+ also significantly declined, proliferating or not (FIG. 17b), suggesting that viruses inhibit this key myogenic transcriptional factor. When measured separately from the myogenic marker Pax7, the cell proliferation, e.g. percent of Ki67+ cells, has also declined upon the control viral transduction (FIG. 17b), which is consistent with the inhibition of p21 by OXTR. Since so few cells were proliferating, it is unlikely that Pax7-cells overtook the culture during this experiment.

These data suggest that viral infections might play a negative role in skeletal muscle maintenance by suppressing myogenic proliferation and may lead to the loss of muscle stem cell identity through down-regulation of Pax7.

### Materials and Methods Examples 7-10

Animal Strain: 22-24 month old C57BL/6 mice were obtained from NIH (Bethesda, Md., USA).

Cell culture: Mouse primary myoblasts were cultured on Matrigel-coated dishes at 37° C., 5% CO2 in growth medium (Ham's F-10, Mediatech); penicillin/streptomycin antibiotics (500 IU/ml, 0.1 mg/ml; MP Biomedicals) and 20% Bovine Growth Serum (Life technologies/Hyclone), supplemented with FGF-2 (6 ng/ml).

Human primary myoblasts were cultured on dilute Matrigel coated dishes (applied at 5 ug/cm\hat{ }2 in phosphate buffered saline, PBS), at 37° C., 5% CO2 in growth medium (Ham's F-10; Mediatech), penicillin/streptomycin antibiotics (500 IU/ml, 0.1 mg/ml; MP Biomedicals) and 5% Bovine Growth Serum (Life technologies/Hyclone), supplemented with FGF-2 (12 ng/ml).

shRNA delivery by lentiviral transduction. In Vivo transduction: All procedures were performed in accordance with the administrative panel of the Office of Laboratory Animal Care, UC Berkeley. Old tibias anterior and gastrocnemius muscles were infected, in vivo, with non-target shRNA (Sigma SHC002V) control transduction lentiviral particles. Mouse Smad3 shRNA-producing lentiviral particles (from Sigma) were used for in vivo transduction experiments (target-set generated from accession number NM_016769.2):

(1) Smad3-targeted shRNA1:
(SEQ ID NO: 10)
CCGGCCCATGTTTCTGCATGGATTTCTCGAGAAATCCATGCAGAAACATG
GGTTTTTG
(2) Smad3-targeted shRNA2:
(SEQ ID NO: 11)
CCGGCCTTACCACTATCAGAGAGTACTCGAGTACTCTCTGATAGTGGTAA
GGTTTTTG
(3) Smad3-targeted shRNA3:
(SEQ ID NO: 12)
CCGGCTGTCCAATGTCAACCGGAATCTCGAGATTCCGGTTGACATTGGAC
AGTTTTTG

Lot: 11191506MN; 7.6×106 -1.4×107 TU/ml stocks were used a MOU 0.3 for each administration of each shRNA-encoding virus. Lentiviral particles were delivered into skeletal muscle by intramuscular injections for two consecutive days and muscle was injured with cardiotoxin on the second day. Three days after the injury, the in vivo infected myofiber-associated satellite cells were isolated, cultured overnight and studied by the RT-PCR.

In Vitro mouse and human primary myoblast transduction. Primary mouse and human myoblasts were transduced in vitro for qRT-PCR, WB, and IF analysis. Primary myoblast cultures were transduced with different MOls (see below) with Opti-MEM (Gibco by Life Technologies). Medium for control groups without any transduction was changed into Opti-MEM. 24 hours later, medium containing lentiviral particles were removed from wells as well as the non-transducer control group, and fresh Opti-MEM were added to each group. Medium were changed every 24 hours until sample collection.

Primary myoblasts were transduced with Smad3-targeted shRNA at MOI=0.5, empty vector (SHC001V) at MOI=0.005, 0.05, 0.5, and with non-target shRNA (SHC002V) at MOI=0.002, 0.02, 0.2, 0.5, 1, 2. Viral titer for lentiviral particles are SHC001V (empty vector control): 7.7E7 TU/ml; SHC002V (non-target shRNA control): 2.6E7 TU/ml

RNA extraction, RT-PCR, and real time PCR: Total RNA was extracted from mouse and human primary myoblast cell culture using RNeasy Mini Kit (QIAGEN) according to manufacturer's instructions. Total RNA was extracted from mouse gastrocnemius and tibialis anterior muscle using QIAshredder (QIAGEN) and RNeasy Mini Kit (QIAGEN) according to manufacturer's instructions. Reverse transcription was performed with Invitrogen Superscript III First-Strand Synthesis System for RT-PCR according to manufacturer's instructions. For real-time PCR amplification and quantification of gene of interest, 1 ug of total cDNA was used for the initial amplification using specific primers to each gene of interest; amplification was performed with a denaturation step at 95° C. for 10 minutes, followed by 40 cycles of denaturation at 95° C. for 15 s and primer extension at 60° C. for 30 s. Real-time PCR was performed using PowerSYBR Green PCR Mastermix (from Applied Biosystems) under QuantStudio3 (Applied Biosystems) and CFX Connect Real-Time System (Bio-Rad). Reactions were run in triplicates. Housekeeping gene GAPDH was used as an internal control to normalize the variability in expression levels and results were analyzed using the 2-ΔΔCT method described.

Primers used in real-time PCR:

(SEQ ID NO: 13)

| | |
|---|---|
| GAPDH1-F: | GGGAAGCCCATCACCATCT |

(SEQ ID NO: 14)

| | |
|---|---|
| GAPDH1-R: | GCCTCACCCCATTTGATGTT |

(SEQ ID NO: 15)

| | |
|---|---|
| GAPDH2-F: | TGAGGCCGGTGCTGAGTATGTCGTG |

(SEQ ID NO: 16)

| | |
|---|---|
| GAPDH2-R: | TCCTTGGAGGCCATGTAGGCCAT |

(SEQ ID NO: 17)

| | |
|---|---|
| Smad3 5′-F: | CTGGCTACCTGAGTGAAGATGGAGA |

(SEQ ID NO: 18)

| | |
|---|---|
| Smad3 5′-R: | AAAGACCTCCCCTCCGATGTAGTAG |

(SEQ ID NO: 19)

| | |
|---|---|
| Smad3 3′-F: | ACACATTGGGAGAGGTGTGC |

(SEQ ID NO: 20)

| | |
|---|---|
| Smad3 3′-R: | GCAAGGGTCCATTCAGGTGT |

(SEQ ID NO: 21)

| | |
|---|---|
| Smad3 shRNA-2-F: | GCCTTACCACTATCAGAGAG |

(SEQ ID NO: 22)

| | |
|---|---|
| Smad3 shRNA-2-R: | AACCTTACCTCATCAGAGAG |

(SEQ ID NO: 23)

| | |
|---|---|
| Smad3 shRNA-3-F: | AACTCTCCAATGTCAACCG |

(SEQ ID NO: 24)

| | |
|---|---|
| Smad3 shRNA-3-R: | GCTGTCCAATGTCAACCG |

(SEQ ID NO: 08)

| | |
|---|---|
| OXTR-F: | GATGTCGCTCGACCGCTG |

(SEQ ID NO: 09)

| | |
|---|---|
| OXTR-R: | CGGTACAATGTAGACGGCGA |

(SEQ ID NO: 25)

| | |
|---|---|
| TGFBR1-F: | TCATTTCAGAGGGCACCACC |

(SEQ ID NO: 26)

| | |
|---|---|
| TGFBR1-R: | CAACTTCTTCTCCCCGCC |

(SEQ ID NO: 27)

| | |
|---|---|
| TGFBR2-F: | TGTATCTTGCCGTTCCCACC |

(SEQ ID NO: 28)

| | |
|---|---|
| TGFBR2-R: | CTCCACAGTGACCACACTCC |

Real-time PCR gel analysis: 4% agarose gel was made using agarose from Fisher Scientific and TAE buffer from Biosciences. 6× loading dye from Fermentas was added to the final amplification product and 15 ul of the mixture was added to the gel. QuickLoad 100 bp DNA Ladder from New England Biolabs was used as reference. Pictures of the gel were taken with a BioRad GelDoc/ChemiDoc Imager and Quantity One software. Pixel density was then analyzed with ImageJ (NIH) by subtracting the background pixel density and normalizing each gene of interest to GAPDH respectively.

Western Blot analysis: Cells were lysed in RIPA buffer containing 1 mM PMSF, 1 mM sodium orthovanadate, PhosSTOP phosphatase inhibitor cocktail (Roche), cOmplete protease inhibitor cocktail (Roche). 30 ug of total protein extract in Laemmli buffer were resolved by SDS-PAGE on 10% precast gels (TGX, Bio-Rad) and transferred to PVDF membranes (Millipore). Membranes were blocked for 1 hr in 5% non-fat milk in PBST at room temperature. Primary antibodies against GAPDH (1:2000), actin (1:1000), pERK1/2 (1:1000), total ERK1/2 (1:1000), pSmad2/3 (1:1000), total Smad2/3 (1:1000), and OXTR (1:1000) were diluted in 5% non-fat milk in PBST. PVDF membranes were incubated in antibody solutions either overnight at 4° C. or 2 hr at room temperature. Horseradish peroxidase-conjugated secondary antibodies were diluted 1:2000 in 1% BSA, and membranes were incubated for 1 hr at room temperature. Blots were developed using ECL reagents (Advansta and Thermo Scientific), and analyzed with Bio-Rad Gel Doc/Chemi Doc Imaging System and Quantity One software. Results of multiple assets were quantified by digitalizing the data and normalizing pixel density of examined proteins by GAPDH pixel density with ImageJ (NIH).

Antibodies
Primary antibodies:
Smad2/3: Cell Signaling Technology #3102
pSmad 2/3: Cell Signaling Technology #8828
ERK1/2: abcam ab184699
pERK1/2: Cell Signaling Technology #9101S
GAPDH: abcam ab9485
beta-actin: (ThermoFisher MA5-15739)
OXTR: proteintech 23045-1-AP
Secondary antibodies (all from Santa Cruz Biotechnology)
Bovine anti-rabbit IgG-HRP: sc-2370
Goat anti-rabbit IgG-HRP: sc-2004
Donkey anti-goat IgG-HRP: sc-2020
Bovine anti-goat IgG-HRP: sc-2350
Bovine anti-mouse IgG-HRP: sc-2371
Goat anti-mouse IgG-HRP: sc-2005
Goat anti-rat IgG-HRP: sc-2006.

Immunofluorescence: Myoblasts were cultured in chamber slides (LabTek CC2 coated glass) and transduced with lentivirus as mentioned above. For OXTR staining, the slides were fixed in cold 4% paraformaldehyde (PFA) on ice for 5 minutes and washed three times in PBS. For Ki67/Pax7 staining, the slides were fixed in 70% cold ethanol at 4° C. overnight. The slides were then permeabilized with 0.25% Triton-100 in PBS for 5 min., and blocked in staining buffer (1% calf serum in PBS) for 2 hr. Primary antibodies were added into staining buffer and incubated for at least 4 hours at room temperature or overnight at 4° C. Three PBS washes were performed before secondary antibody were added and incubated overnight at 4° C. Slides were then washed three times with PBS before mounted for fluorescence imaging.

Secondary antibodies: Invitrogen Alexa Fluor 488 Donkey anti-Rabbit IgG (H+L) Secondary Antibody. Invitrogen Alexa Fluor 488 Goat anti-Mouse IgG (H+L) Secondary Antibody.

GEO Dataset analysis: Patient studies for viral infections were identified from Gene Expression Omnibus datasets (GEO https://www.ncbi.nlm.nih.gov/geo/). Every dataset with information on OXTR, TGFBR1, or TGFBR2 were analyzed, regardless of cell types and types of viral infection. The fold changes of each gene measured in patients were normalized to non-infected control groups in a human study. For datasets with time-course experiments, time points starting and after 72 hours were calculated. The accessions of GEO datasets used are as follows:

### GSE18816, GSE23031, GSE16593, GSE23031, GSE2067, GSE2067, GSE3292, GSE13597, GSE6802, GSE2815, GSE48466, GSE24533, GSE3397, GSE22589, GSE49954, GSE18816, GSE4785, GSE20755, GSE20948, GSE3397, GSE30719, GSE27131.

Data Quantification: A non-paired, 2-tailed T-test was performed on all of the respective data. Quantified data were presented as means (SD). P-Values of <0.05 were considered statistically significant.

what is claimed is:

1. A method of treating a subject with a viral infection, the method comprising:
administering an effective amount of an oxytocin receptor (OXTR) agonist and an ALK5 antagonist,
wherein the effective amount of OXTR agonist and ALK5 antagonist enhances OXTR expression in the subject.

2. The method of claim 1, wherein the subject is a human.

3. The method of any of the previous claims, wherein the subject is an animal.

4. The method of any of the previous claims, wherein the viral infection is an acute viral infection.

5. The method of any of the previous claims, wherein the viral infection is a chronic viral infection.

6. The method of any of the previous claims, wherein the effective amount of an oxytocin receptor (OXTR) agonist and an ALK5 antagonist are administered simultaneously.

7. The method of any of the previous claims, wherein the subject with a viral infection is an aged subject.

8. The method of any of the previous claims, wherein the amount of the OXTR agonist is in the range of 7.5 nM-30 nM.

9. The method of any of the previous claims, wherein the amount of the ALK5 antagonist is in the range of 0.05 μM-3 μM.

10. The method of any of the previous claims, wherein the ratio of the OXTR agonist to the ALK5 antagonist is 1:50.

11. The method of any of the previous claims, wherein the ratio of the OXTR agonist to the ALK5 antagonist is 50:1.

12. The method of any of the previous claims, wherein the ratio of the OXTR agonist to the ALK5 antagonist is 1:40.

13. The method of any of the previous claims, wherein the ratio of the OXTR agonist to the ALK5 antagonist is 1:40.

14. The method of any of the previous claims, wherein the ratio of the OXTR agonist to the ALK5 antagonist is 40:1.

15. The method of any of the previous claims, wherein the ratio of the OXTR agonist to the ALK5 antagonist is 1:25.

16. The method of any of the previous claims, wherein the ratio of the OXTR agonist to the ALK5 antagonist is 25:1.

17. The method of any of the previous claims, wherein the ratio of the OXTR agonist to the ALK5 antagonist is 1:10.

18. The method of any of the previous claims, wherein the ratio of the OXTR agonist to the ALK5 antagonist is 10:1.

19. The method of any of the previous claims, wherein the ratio of the OXTR agonist to the ALK5 antagonist is 1:5.

20. The method of any of the previous claims, wherein the ratio of the OXTR agonist to the ALK5 antagonist is 5:1.

21. The method of any of the previous claims, wherein the ratio of the OXTR agonist to the ALK5 antagonist is 1:1.

22. The method of any of the previous claims, wherein the OXTR agonist is oxytocin.

23. The method of any of the previous claims, wherein the ALK5 antagonist is 2-(3-(6-Methylpyridin-2-yl)-1H-pyrazol-4-yl)-1,5-naphthyridine.

24. The method of any of the previous claims, wherein the OXTR expression in the subject is as compared to the OXTR expression from a healthy adult subject.

25. The method of any of the previous claims, further comprising assessing the OXTR expression in the subject following the administration and adjusting the amount of the OXTR agonist and/or the ALK5 antagonist.

26. The method of any of the previous claims, further comprising assessing the OXTR expression in the subject following the administration and decreasing the amount of the OXTR agonist.

27. The method of any of the previous claims, further comprising assessing the OXTR expression in the subject following the administration and increasing the amount of the ALK5 antagonist.

28. The method of any of the previous claims, further comprising assessing the OXTR expression in the subject following the administration and decreasing the amount of the ALK5 antagonist.

29. The method of any of the previous claims, further comprising assessing the OXTR expression in the subject following the administration and repeating the administration on a schedule.

30. The method of claim 29, further comprising assessing the OXTR expression in the subject following the repeated administration and adjusting the contacting schedule.

31. A method of enhancing hippocam pal neurogenesis in a subject, the method comprising:
administering an effective amount of an oxytocin receptor (OXTR) agonist and an ALK5 antagonist,
wherein the effective amount of OXTR agonist and ALK5 antagonist reduces CD45+ cells in the brain of the subject.

32. The method of claim 31, wherein the subject is an aged subject.

33. The method of claim 31 or claim 32, wherein the hippocam pal neurogenesis in the subject exhibits a two-fold increase within 1 week.

34. The method of claim 32, wherein the CD45+ cells in the brain of the aged subject are elevated as compared to the CD45+ cells in the healthy brain of a young subject.

35. A method of enhancing functional learning in a subject, the method comprising:
administering an effective amount of an oxytocin receptor (OXTR) agonist and an ALK5 antagonist,
wherein the effective amount of OXTR agonist and ALK5 antagonist improves memory and cognition in the subject.

36. A method of enhancing liver regeneration and reducing liver fibrosis and adiposity in a subject, the method comprising:
administering an effective amount of an oxytocin receptor (OXTR) agonist and an ALK5 antagonist,
wherein the effective amount of OXTR agonist and ALK5 antagonist improves liver regeneration and reduces liver fibrosis and adiposity in the subject.

37. A method of reducing p16 in at least one tissue of a subject, the method comprising:
administering an effective amount of an oxytocin receptor (OXTR) agonist and an ALK5 antagonist.

38. The method of claim 37, wherein at least one tissue is an old muscle.

39. The method of claim 37, wherein at least one tissue is the liver of the subject.

40. The method of claim 37, wherein at least one tissue is the brain of the subject.

41. The method of claim 37, wherein the p16 is reduced in at least one tissue of the subject as compared to the p16 of the same type of tissue from a younger subject.
